SEQUENCE LISTING (16 sequences)

SEQ ID NO: 1 - protein, 124 aa, Synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 2 - protein, 124 aa, Synthetic construct
  MOD_RES 109: note = X is fluorosulfate-L-tyrosine (FSY)
QVKLEESGGGSVQTGGSLRLTCAASGRTSRSYGMGWFRQAPGKRREFVSGISWRGDSTGYADSVKGRFTISRDNAKNTVDLQMNSLKPEDTAIYYCAAAAGSAWYGTLXEYDYWGQGTQVTVSS

SEQ ID NO: 3 - protein, 9 aa, Synthetic construct
RTSRSYGMG

SEQ ID NO: 4 - protein, 8 aa, Synthetic construct
GISWRGDS

SEQ ID NO: 5 - protein, 15 aa, Synthetic construct
AAGSAWYGTLYEYDY

SEQ ID NO: 6 - protein, 15 aa, Synthetic construct
  MOD_RES 11: note = X is fluorosulfate-L-tyrosine (FSY)
AAGSAWYGTLXEYDY

SEQ ID NO: 7 - protein, 14 aa, Synthetic construct
WFRQAPGKRREFVS

SEQ ID NO: 8 - protein, 115 aa, Synthetic construct
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 9 - protein, 115 aa, Synthetic construct
  MOD_RES 109: note = X is fluorosulfate-L-tyrosine (FSY)
QVQLQESGGGSVQAGGSLKLTCAASGYIFNSCGMGWYRQSPGRERELVSRISGXGRTWHKESVKGRFTISQDNVKKTLYLQMNSLKPEDTAVYFCAVCYNLETYWGQGTQVTVSS

SEQ ID NO: 10 - protein, 8 aa, Synthetic construct
GYIFNSCG

SEQ ID NO: 11 - protein, 7 aa, Synthetic construct
RISGDGD

SEQ ID NO: 12 - protein, 9 aa, Synthetic construct
AVCYNLETY

SEQ ID NO: 13 - protein, 7 aa, Synthetic construct
  MOD_RES 5: note = X is fluorosulfate-L-tyrosine (FSY)
RISGXGR

SEQ ID NO: 14 - protein, 58 aa, Synthetic construct
VDNKFNKELSVAGREIVTLPNLNDPQKKAFIFSLWDDPSQSANLLAEAKKLNDAQAPK

SEQ ID NO: 15 - protein, 58 aa, Synthetic construct
  MOD_RES 36: note = X is fluorosulfate-L-tyrosine (FSY)
VDNKFNKELSVAGREIVTLPNLNDPQKKAFIRSLWXDPSQSANLLAEAKKLNDAQAPK

SEQ ID NO: 16 - protein, 127 aa, Synthetic construct
QVQLQESGGGSVQTGGSLRLSCAASGYTASFSWIGYFRQAPGKEREGVAVINVGVGSTYYADSVKGRFTISRDNTENTISLEMNSLKPEDTGLYYCAGSLRWSRPPNPISEDAYNYWGQGTQVTVSS

(19) United States
(12) Patent Application Publication
(10) Pub. No.: US 2026/0347482 A1
(43) Pub. Date: Nov. 5, 2026

(54) BIOREACTIVE PROTEINS CONTAINING AN UNNATURAL AMINO ACID AND ARGININE

(71) Applicant: The Regents of the University of California, Oakland, CA (US)

(72) Inventors: Lei Wang, San Francisco, CA (US); Li Cao, San Francisco, CA (US)

(21) Appl. No.: 19/143,102

(22) PCT Filed: Jan. 2, 2024
(86) PCT No.: PCT/US2024/010060

§ 371 Date: Jun. 25, 2025

Related U.S. Application Data
(60) Provisional application No. 63/436,345, filed on Dec. 30, 2022.

(51) Int. Cl. C07K 16/28

(52) U.S. Cl. CPC C07K 16/2863; C07K 2317/565; C07K 2317/569; C07K 2317/92

(57) ABSTRACT

Provided herein are, inter alia, proteins comprising unnatural amino acids and arginine, pharmaceutical compositions comprising the proteins, and methods of enhancing the bioreactivity of proteins. In embodiments, the unnatural amino comprises a side chain of Formula (I), wherein the substituents are as defined in the specification.

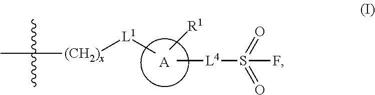

### CROSS-REFERENCE TO RELATED APPLICATION

This application claims the benefit of priority to U.S. Application No. 63/436,345 filed Dec. 30, 2022, the disclosure of which is incorporated by reference herein.

### STATEMENT AS TO RIGHTS TO INVENTIONS MADE UNDER FEDERALLY SPONSORED RESEARCH AND DEVELOPMENT

This invention was made with government support under R01 GM118384 awarded by the National Institutes of Health. The government has certain rights in the invention.

### REFERENCE TO A SEQUENCE LISTING, A TABLE, OR A COMPUTER PROGRAM LISTING APPENDIX SUBMITTED AS AN ASCII FILE

The Sequence Listing entitled 048536-760001WO-Sequence Listing, was created in XML format on IBM-PC, MS Windows operating system on Dec. 20, 2023, and having 17,633 bytes is hereby incorporated by reference.

### BACKGROUND

Proximity-enabled sulfur fluoride exchange reaction has been increasingly used to covalently crosslink proteins and to generate covalent protein drugs. Such reaction is made possible by genetically incorporating into proteins latent bioreactive unnatural amino acids (Uaas) that bear either fluorosulfonate or sulfonyl fluoride functional groups. For many applications, a critical requirement for these reactions is the fast reaction rate, so that more proteins can be covalently crosslinked in a short contact time, which can increase the sensitivity of protein detection or enhance the potency of protein drugs. Provided herein, inter alia, are solutions to these and other needs in the art.

### SUMMARY

Provided herein are proteins comprising: (i) an unnatural amino acid, and (ii) a non-naturally occurring arginine; wherein: (a) the non-naturally occurring arginine is proximal to the —S(O₂)F group in the unnatural amino acid side chain; and (b) the unnatural amino comprises a side chain of Formula (I)

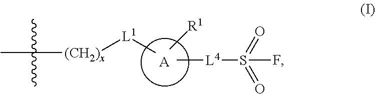

where the substituents are defined herein. In embodiments, the protein is an antibody or an antibody variant, such as a single-chain variable fragment, a single-domain antibody, an affibody, or an antigen-binding fragment.

Provided herein are proteins comprising: (i) an unnatural amino acid, and (ii) arginine; wherein: (a) the arginine is proximal to the —S(O₂) F group in the unnatural amino acid side chain; and (b) the unnatural amino comprises a side chain of Formula (I):

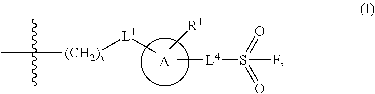

where the substituents are defined herein. In embodiments, the protein is an antibody or an antibody variant, such as a single-chain variable fragment, a single-domain antibody, an affibody, or an antigen-binding fragment.

These and other embodiments of the disclosure are provided in detail herein.

### BRIEF DESCRIPTION OF THE DRAWINGS

FIGS. 1A-1D show binding of nanobody 7D12 with EGFR. FIG. 1A: Crystal structure of nanobody 7D12-EGFR ECD showing Y109 for FSY incorporation, target residue K443, and E44 mutated to R. FIG. 1B: SDS-PAGE analysis of nanobody 7D12 (Y109FSY) at left and 7D12 (Y109FSY/E44R) at right crosslinking with EGFR ECD protein. FIG. 1C: SDS-PAGE analysis of nanobody 7D12 (Y109FSY) at left and 7D12 (Y109FSY/T107R) at right crosslinking with EGFR ECD protein. FIG. 1D: SDS-PAGE analysis of nanobody 7D12 (Y109FSY) at left and 7D12 (Y109FSY/L108R) at right crosslinking with EGFR ECD protein.

FIGS. 2A-2B show binding of nanobody 2Rs15d to HER2. FIG. 2A: Crystal structure of nanobody 2Rs15d-HER2 ECD showing D54 for FSY incorporation, target residue K150, and D56 mutated to R. FIG. 2B: SDS-PAGE analysis of nanobody 2Rs15d (D54FSY) at left and 2Rs15d (D54FSY/D56R) at right crosslinking with HER2 ECD protein.

FIGS. 3A-3D show binding of affibody ZSPA to protein Z pair. FIG. 3: Crystal structure of affibody-Z complex showing D36 for FSY incorporation, N6 mutating to H/K/Y, and F32 mutated to R. FIG. 3B: SDS-PAGE analysis of affibody (36FSY) at left and affibody (36FSY/32R) at right crosslinking with MBP-Z (N6H) protein. FIG. 3C: SDS-PAGE analysis of affibody (36FSY) at left and affibody (36FSY/32R) at right crosslinking with MBP-Z (N6K) protein. FIG. 3D: SDS-PAGE analysis of affibody (36FSY) at left and affibody (36FSY/32R) at right crosslinking with MBP-Z (N6Y) protein.

FIG. 4 is an SDS-PAGE analysis of nanobody Nb13 (G54FSY) at left and nanobody Nb13 (G54FSY/55R) at right crosslinking with PSMA ECD protein.

FIG. 5 is an SDS-PAGE analysis of nanobody 7D12 (Y109mFSY) at left and nanobody 7D12 (Y109mFSY/E44R) at right crosslinking with EGFR ECD protein.

FIG. 6 is an SDS-PAGE analysis of nanobody 2Rs15d (D54FFY) at left and nanobody 2Rs15d (D54FFY/D56R) at right crosslinking with HER2 ECD protein.

FIG. 7 is an SDS-PAGE analysis of nanobody 7D12 (Y109FFY) at left and nanobody 7D12 (Y109FFY/E44R) at right crosslinking with EGFR ECD protein.

FIG. 8 is an SDS-PAGE analysis of WT BiKE 7D12 (Y109FSY)-C21 at left and Arg-BIKE 7D12 (Y109FSY/E44R)-C21 at right crosslinking with EGFR ECD protein.

FIG. 9 is a Western blot analysis of WT BiKE 7D12 (Y109FSY)-C21 at left and Arg-BiKE 7D12 (Y109FSY/E44R)-C21 at right crosslinking with EGFR on the surface of A431 cells.

FIG. 10 shows perforin level in NK-A431 cell incubation with A431 cell pre-treated with different BiKEs.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The term “sulfur-fluoride exchange reaction” or “SuFEx” refers to a type of click chemistry as described in detail by, e.g., Dong et al, Angewandte Chemie, 53 (36): 9340-9448 (2014); and Wang et al, J. Am. Chem. Soc., 140 (15): 4995-4999 (2018). The term “proximally-enabled” SuFEx refers to the sulfur-fluoride exchange reaction occurring when the reactive species are proximal to each other, i.e., spatially close enough for the SuFEx reaction to occur. The skilled artisan could readily determine whether the reactive species are sufficiently proximal for the reaction to occur, e.g., sulfur-fluoride exchange reaction between a protein comprising an unnatural amino acid having a side chain of Formula (I) and a target protein (e.g., a protein having a tyrosine, lysine, or histidine). Without intending to be bound by any theory, the arginine (either non-naturally occurring arginine or naturally occurring arginine) side chain interacts through van der Waals forces with the —S(O₂) F group in the unnatural amino acid side chain in the protein. When the protein contacts the target protein, the arginine side chain in the protein facilitates the —F departure from the —S(O₂) F group in the unnatural amino acid side chain, which enhances the SuFEx reaction in which the resulting —S(O₂)— group in the protein covalently bonds with lysine, histidine, or tyrosine in the target protein. “Enhances” or “enhancing” refers to increasing the reaction rate and/or the yield of the protein covalently binding to the target protein.

“Arginine” refers to the amino acid having the following structure:

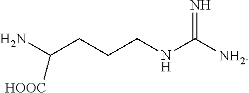 [SMILES: N=C(N)NCCCC(N)C(=O)O]

Arginine has the following side chain:

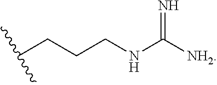 [SMILES: *CCCNC(=N)N]

As used herein, a “non-naturally occurring arginine” and a “naturally occurring arginine” are structurally and chemically the same.

The term “non-naturally occurring arginine” refers to an arginine that is a point mutation of a different amino acid (e.g., Ala, Ile, Leu, Met, Val, Phe, Trp, Tyr, Asn, Cys, Gln, Ser, Thr, Asp, Glu, His, Lys, Gly, Pro) that naturally occurs in a protein. For example, SEQ ID NO: 2 contains a naturally occurring arginine at position 45 (e.g., with reference to wild-type SEQ ID NO:1, the corresponding amino acid at position 45 is arginine) and contains a non-naturally arginine at position 44 (e.g., with reference to wild-type SEQ ID NO:1, the corresponding amino acid at position 44 is glutamic acid). In embodiments, the “non-naturally occurring arginine” is proximal in a three-dimensional space to an unnatural amino acid having a side chain comprising an −S(O₂) F group in the same protein, such that the non-naturally occurring arginine can interact through van der walls forces with the −S(O₂) F group in the unnatural amino acid side chain.

The term “naturally-occurring arginine” refers to an arginine that naturally occurs at a given position in a protein. In embodiments, the “naturally occurring arginine” is proximal in a three-dimensional space to an unnatural amino acid having a side chain comprising an −S(O₂) F group in the same protein, such that the naturally occurring arginine can interact through van der walls forces with the —S(O₂) F group in the unnatural amino acid side chain.

The term “proximal” means that an arginine (either non-naturally occurring arginine or naturally occurring arginine) is of a relationship (e.g., distance) that it can interact through van der Waals forces with the —S(O₂) F group in an unnatural amino acid side chain of an unnatural amino acid, as described herein (including embodiments thereof). In embodiments, the “proximal” means that an arginine (either non-naturally occurring arginine or naturally occurring arginine) can facilitate the —F departure from the —S(O₂) F group in an unnatural amino acid side chain of an unnatural amino acid when the —S(O₂) F group reacts with the lysine, histidine, or tyrosine of a target protein through a SuFEx reaction. The skilled artisan will readily appreciate that “proximal” is based on the three-dimensional structure of the protein. In embodiments, “proximal” means up to about 25 angstroms. In embodiments, “proximal” means up to about 20 angstroms. In embodiments, “proximal” means up to about 15 angstroms. In embodiments, “proximal” means up to about 10 angstroms. In embodiments, “proximal” means from about 1 angstrom to about 25 angstroms. In embodiments, “proximal” means from about 1 angstrom to about 20 angstroms. In embodiments, “proximal” means from about 1 angstrom to about 15 angstroms. In embodiments, “proximal” means from about 1 angstrom to about 12 angstroms. In embodiments, “proximal” means from about 1 angstrom to about 10 angstroms. In embodiments, “proximal” means from about 1 angstrom to about 8 angstroms. In embodiments, “proximal” means from about 1 angstrom to about 6 angstroms. In embodiments, “proximal” means from about 1 angstrom to about 5 angstroms. In embodiments, “proximal” means from about 1 angstroms to about 4 angstroms.

In embodiments, the term “proximal” means that the naturally or non-naturally occurring arginine is within 1 to about 10 amino acid residues of the unnatural amino acid. In embodiments, the naturally or non-naturally occurring arginine is within 1 to about 9 amino acid residues of the unnatural amino acid. In embodiments, the naturally or non-naturally occurring arginine is within 1 to about 8 amino acid residues of the unnatural amino acid. In embodiments, the naturally or non-naturally occurring arginine is within 1 to about 7 amino acid residues of the unnatural amino acid. In embodiments, the naturally or non-naturally occurring arginine is within 1 to about 6 amino acid residues of the unnatural amino acid. In embodiments, the naturally or non-naturally occurring arginine is within 1 to about 5 amino acid residues of the unnatural amino acid. In embodiments, the naturally or non-naturally occurring arginine is within 1 to about 4 amino acid residues of the unnatural amino acid. In embodiments, the naturally or non-naturally occurring arginine is within 1 to about 3 amino acid residues of the unnatural amino acid. In embodiments, the naturally or non-naturally occurring arginine is within 1 to about 2 amino acid residues of the unnatural amino acid. In embodiments, the naturally or non-naturally occurring arginine is within 2 to about 6 amino acid residues of the unnatural amino acid. In embodiments, the naturally or non-naturally occurring arginine is within 2 to about 5 amino acid residues of the unnatural amino acid. In embodiments, the naturally or non-naturally occurring arginine is within 2 to about 4 amino acid residues of the unnatural amino acid. In embodiments, the naturally or non-naturally occurring arginine is within 2 to about 3 amino acid residues of the unnatural amino acid. In embodiments, the naturally or non-naturally occurring arginine is within 1 amino acid residue of the unnatural amino acid. In embodiments, the naturally or non-naturally occurring arginine is within 2 amino acid residues of the unnatural amino acid. In embodiments, the naturally or non-naturally occurring arginine is within 3 amino acid residues of the unnatural amino acid. In embodiments, the naturally or non-naturally occurring arginine is within 4 amino acid residues of the unnatural amino acid. In embodiments, the naturally or non-naturally occurring arginine is within 5 amino acid residues of the unnatural amino acid. The phrase “within 1” means that the naturally or non-naturally-occurring arginine is next to the unnatural amino acid. The phrase “within 2” means that there is one amino acid residue between the naturally or non-naturally-occurring arginine and the unnatural amino acid.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F (ab)′2, a dimer of Fab which itself is a light chain joined to VH-CHI by a disulfide bond. The term “F (ab)′2” is used interchangeably with “Fab dimer.” The F (ab)′2 may be reduced under mild conditions to break the disulfide linkage in the hinge region, thereby converting the F (ab)′2 dimer into an Fab′ monomer. The Fab′ monomer is essentially Fab with part of the hinge region (see Fundamental Immunology (Paul ed., 3d ed. 1993)). The term “Fab′ monomer” is used interchangeably with “Fab” and “or an antigen-binding fragment.” While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (e.g., McCafferty et al., Nature 348:552-554 (1990)).

Antibodies are large, complex proteins with an intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat et al, Sequences of Proteins of Immunological Interest, U.S. Department of Health and Human Services, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” and one “heavy” chain. The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

An “antibody variant” as provided herein refers to a polypeptide capable of binding to a receptor protein or an antigen and including one or more structural domains of an antibody or fragment thereof. Non-limiting examples of antibody variants include single-domain antibodies (nanobodies), affibodies (polypeptides smaller than monoclonal antibodies and capable of binding receptor proteins or antigens with high affinity and imitating monoclonal antibodies), antigen-binding fragments (Fab), Fab dimers (monospecific Fab₂, bispecific Fab₂), trispecific Fab₃, monovalent IgGs, single-chain variable fragments (scFv), bispecific diabodies, trispecific triabodies, scFv-Fc, minibodies, IgNAR, V-NAR, hclgG, VhH, and peptibodies. A “peptibody” refers to a peptide moiety attached (through a covalent or non-covalent linker) to the Fc domain of an antibody.

A “single-domain antibody” or “nanobody” refers to an antibody fragment having a single monomeric variable antibody domain. Like a whole antibody, it is able to bind selectively to a specific antigen. In embodiments, the single domain antibody is a human or humanized single-domain antibody.

A single-chain variable fragment (scFv) is typically a fusion protein of the variable regions of the heavy (VH) and light chains (VL) of immunoglobulins, connected with a short linker peptide of 10 to about 25 amino acids. The linker is usually rich in glycine for flexibility, as well as serine or threonine for solubility. The linker can either connect the N-terminus of the VH with the C-terminus of the VL, or vice versa.

Antibodies, e.g., recombinant, monoclonal, or polyclonal antibodies, can be prepared by techniques well known in the art. The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies can be adapted to produce antibodies to polypeptides. Also, transgenic mice, or other organisms such as other mammals, may be used to express humanized or human antibodies. Alternatively, phage display technology can be used to identify antibodies and heteromeric Fab fragments that specifically bind to selected antigens. Antibodies can also be made bispecific, i.e., able to recognize two different antigens. Antibodies can also be heteroconjugates, e.g., two covalently joined antibodies, or immunotoxins.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent CDRs or CDR sequences for the corresponding sequences of a human antibody. Accordingly, such humanized antibodies are chimeric antibodies, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (i) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (ii) the variable region, or a portion thereof, is altered, replaced or exchanged with a variable region having a different or altered antigen specificity. In embodiments, the antibodies described herein include humanized and/or chimeric monoclonal antibodies.

The phrase “specifically (or selectively) binds” to an antibody or a receptor protein or “specifically (or selectively) immunoreactive with” when referring to a protein refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (e.g., Harlow & Lane, Using Antibodies, A Laboratory Manual (1998) for a description of immunoassay formats and conditions that can be used to determine specific immunoreactivity).

The term “target protein” refers to a targeting molecule having, e.g., a regulatory role in a cell. In embodiments, a “target protein” is a receptor protein, a cytosolic protein, a transcriptional factor, or an enzyme. In embodiments, a receptor protein is an extracellular domain receptor protein, a transmembrane domain receptor protein, or an intracellular domain receptor protein. In embodiments, the “target protein” is the binding target of the antibody or antibody variant described herein. In embodiments, the protein (e.g., antibody or antibody variant) described herein is capable of inhibiting or activating the biological activity of the target protein upon binding. In embodiments, the activity of the target protein is increased or decreased.

“Receptor protein” or “membrane receptor” refers to a receptor (protein) that is embedded in the plasma membrane of a cell. In embodiments, the receptor protein is located in the extracellular domain of a cell, the transmembrane domain of a cell, or the intracellular domain of a cell. In embodiments, the receptor protein is a cell-surface receptor. In embodiments, the receptor protein is in the extracellular domain. In embodiments, the receptor protein is in the transmembrane domain. In embodiments, the receptor protein is an ion channel-linked receptor, an enzyme-linked receptor, or a G protein-coupled receptor. In embodiments, the receptor protein is a hormone receptor.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and unnaturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Glycan Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides may be referred to by their commonly accepted single-letter codes.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain.

The term “unnatural amino acid” refer to amino acid analogs, synthetic amino acids, and amino acid mimetics which are not found in nature. In embodiments, the term “unnatural amino acid” refers to a compound having the structure of Formula (A):

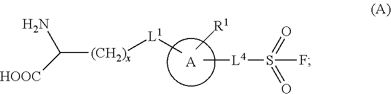

wherein the substituents are as defined herein.

The term “FSY” refers to a compound having the structure of Formula (FSY):

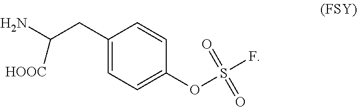 [SMILES: NC(Cc1ccc(OS(=O)(=O)F)cc1)C(=O)O]

An amino acid having an “FSY side chain” refers to the following side chain:

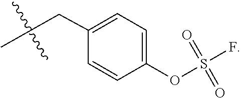 [SMILES: *Cc1ccc(OS(=O)(=O)F)cc1]

The term “FFY” refers to a compound having the structure of Formula (FFY)

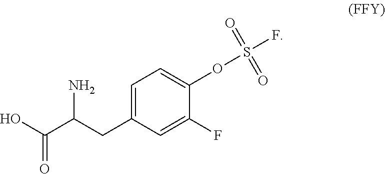 [SMILES: NC(Cc1ccc(OS(=O)(=O)F)c(F)c1)C(=O)O]

The term “mFSY” refers to a compound having the structure of Formula (mFSY)

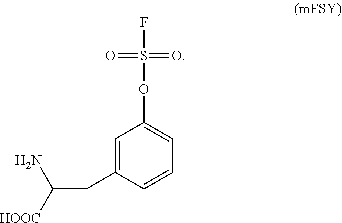 [SMILES: NC(Cc1cccc(OS(=O)(=O)F)c1)C(=O)O]

The term “unnatural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Unnatural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. In embodiments, the term unnatural amino side chain refers to a moiety having Formula (I):

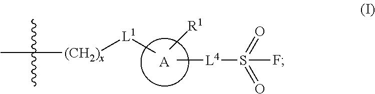

where the substituents are as defined herein.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The terms “protein,” “polypeptide,” and “peptide” are used interchangeably herein to refer to a polymer of amino acid residues. The polymer of amino acids may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and unnaturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to specific position (e.g., A100) of a protein when the selected residue occupies the same essential spatial or other structural relationship as that specific position (e.g., A100) of the protein. In embodiments, where a selected protein is aligned for maximum homology with the protein, the position in the aligned selected protein aligning with that specific position (e.g., A100) is said to correspond to that specific residue (e.g., A100). Instead of a primary sequence alignment, a three dimensional structural alignment can also be used, e.g., where the structure of the selected protein is aligned for maximum correspondence with the protein and the overall structures compared. In this case, an amino acid that occupies the same essential position as that specific position (e.g., A100) in the structural model is said to correspond to the that specific position residue (e.g., A100).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, or at least 65%, 70%, 75%, 80%, 85%, 90%, 91%, 92%, 93%, 94%, 95%, 96%, 97%, 98%, 99% identity over a specified region, when compared and aligned for maximum correspondence over a comparison window or designated region) as measured using a BLAST or BLAST 2.0 sequence comparison algorithms with default parameters described below, or by manual alignment and visual inspection (e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “vector” refers to a nucleic acid molecule capable of transporting another nucleic acid to which it has been linked. One type of vector is a “plasmid,” which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. The terms “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions. Some viral vectors are capable of targeting a particular cells type either specifically or non-specifically. Exemplary vectors that can be used include, but are not limited to, pEvol vector, pMP vector, pET vector, pTak vector, pBad vector.

The terms “transfection,” “transduction,” “transfecting,” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

“Contacting” is used in accordance with its plain ordinary meaning and refers to the process of allowing at least two distinct species (e.g., protein and a target protein) to become sufficiently proximal to react, interact, or physically touch. It should be appreciated that the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be proteins as described herein.

The term “intermolecular linker” refers to a linking group between two proteins.

The term “a protein described in WO 2017/161183” refers to any protein, including, without limitation, an antibody or an antibody variant, described in WO 2017/161183. In embodiments, “a protein described in WO 2017/161183” is mouse double minute 2 homolog (MDM2), protein MDM4, and protein 53 (p53). The amino acid sequence of each of these proteins is set forth in WO 2020/072674 or is known in the art.

The term “a protein described in WO 2019/173760” refers to any protein, including, without limitation, an antibody or an antibody variant, described in WO 2019/173760. In embodiments, “a protein described in WO 2019/173760” is enhanced green fluorescent protein (EGFP), ZSPA affibody, maltose binding protein, protein Z, thioredoxin (TRX1), and human growth hormone. The amino acid sequence of each of these proteins is set forth in WO 2020/072674 or is known in the art.

The term “a protein described in WO 2020/072674” refers to any protein, including, without limitation, an antibody or an antibody variant, described in WO 2020/072674. In embodiments, “a protein described in WO 2020/072674” is bovine serum albumin, peptide 7KR, glutathione S-transferase, 14-3-3 protein, and single-strand DNA binding protein (SSB). The amino acid sequence of each of these proteins is set forth in WO 2020/072674 or is known in the art.

The term “a protein described in WO 2020/206341” refers to any protein, including, without limitation, an antibody or an antibody variant, described in WO 2020/206341. In embodiments, “a protein described in WO 2020/206341” is ZSPA affibody, enhanced green fluorescent protein (EGFP), and Z protein. The amino acid sequence of each of these proteins is set forth in WO 2020/206341 or is known in the art.

The term “a protein described in WO 2022/232377” refers to any protein, including, without limitation, an antibody or an antibody variant, described in WO 2022/232377. In embodiments, “a protein described in WO 2022/232377” is a CRISPR protein, Hfq, ACE2 receptor protein, SARS-COV-2 spike(S) protein, SR4 nanobody, MR17K99Y nanobody, nanobody H11D4, mNb6 nanobody, nanobody 2rs15d (NbHER2), nanobody C21, nanobody NB13, nanobody NB17B05, MS211, ZHER2: 2891, ZHER2: 342, F57 (or 5F7), nanobody 7D12 (Nb_{EGFR}), trastuzumab, trastuzumab Fab, neuregulin 1β (NRG1b), thioredoxin (TRX), NK035, A1 nanobody, and C6 nanobody. The amino acid sequence of each of these proteins is set forth in WO 2022/232377 or is known in the art.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified by, e.g., —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) may be placed at any interior position of the heteroalkyl group or at the position at which the alkyl group is attached to the remainder of the molecule. Heteroalkyl is an uncyclized chain. Examples include, but are not limited to: —CH₂NH₂, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—NH₂, —CH₂—NO₂, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms. A heteroalkyl moiety may include five optionally different heteroatoms. A heteroalkyl moiety may include up to 8 optionally different heteroatoms. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′-represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl. In embodiments of the compounds of Formula (I), Formula (II), and Formula (III) described herein (including embodiments thereof), ring A is a 5-membered monocyclic cycloalkyl, a 5-membered monocyclic heterocycloalkyl, or a 5-membered monocyclic heteroaryl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂) w, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl. In embodiments of the compounds of Formula (I), Formula (II), and Formula (III) described herein (including embodiments thereof), ring A is a 5-membered monocyclic cycloalkyl, a 5-membered monocyclic heterocycloalkyl, or a 5-membered monocyclic heteroaryl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl. In embodiments of the compounds of Formula (I), Formula (II), and Formula (III) described herein (including embodiments thereof), ring A is a 5-membered monocyclic cycloalkyl, a 5-membered monocyclic heterocycloalkyl, or a 5-membered monocyclic heteroaryl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo (C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen. In embodiments of the compounds of Formula (I), Formula (II), and Formula (III) described herein (including embodiments thereof), ring A is a 5-membered monocyclic cycloalkyl, a 5-membered monocyclic heterocycloalkyl, or a 5-membered monocyclic heteroaryl.

The symbol “~” or “-” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker).

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, —NR′, ═N—OR′, —NR′R″, -SR′, -halogen, —SiR′R″R″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R′″)═NR″, —NR—C(NR′R″)═NR″, —S(O)R′, —S(O)₂R′, —S(O)₂NR′R″, —NRSO₂R′, —NR′NR″R″, —ONR′R″, —NR′C(O)NR″NR″R″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″, and R″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O) CH₃, —C(O) CF₃, —C(O) CH₂OCH₃, and the like).

Similar to the substituents described for the alkyl radical, substituents for the aryl and heteroaryl groups are varied and are selected from, for example:—OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R″, —NR″C(O)₂R′, —NR—C(NR′R″R″)═NR″, —NR—C(NR′R″)═NR″, —S(O)R′, —S(O)₂R′, —S(O)₂NR′R″, —NRSO₂R′, —NR′NR″R″, —ONR′R″, —NR′C(O)NR″NR″R″, —CN, —NO₂, —R′, —N₃, —CH(Ph)₂, fluoro (C₁-C₄)alkoxy, and fluoro (C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R″, and R″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″, and R″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In embodiments, the ring-forming substituents are attached to non-adjacent members of the base structure.

Two of the substituents on adjacent atoms of the aryl or heteroaryl ring may optionally form a ring of the formula —T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula —A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ-X′-(C″R″R′″)_{d}-, where s and d are independently integers of from 0 to 3, and X′ is —O—, —NR′—, —S—, —S(O)—, —S(O)₂—, or —S(O)₂NR′—. The substituents R, R′, R″, and R″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur(S), phosphorus (P), and silicon (Si).

A “substituent group,” as used herein, means a group selected from the following moieties: (A) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC₁₂, —OCHBr₂, —OCHI₂, —OCHF2, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (B) alkyl, heteroalkyl, cycloalkyl, heterocycloalkyl, aryl, heteroaryl, substituted with at least one substituent selected from: (i) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCI₂, —OCHBr₂, —OCHI2, —OCHF2, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl, heteroalkyl, cycloalkyl, heterocycloalkyl, aryl, heteroaryl, substituted with at least one substituent selected from: (a) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O) H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O) H, —NHC(O)OH, —NHOH, —OCCl₃, —OCF₃, —OCBr₃, —OCI₃, —OCHC12, —OCHBr₂, —OCHI₂, —OCHF2, unsubstituted alkyl (e.g., C₁-C₈alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, in embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In embodiments, at least one or all of these groups are substituted with at least one lower substituent group.

In embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds described herein possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or(S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and(S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers. As used herein, the term “isomers” refers to compounds having the same number and kind of atoms, and hence the same molecular weight, but differing in respect to the structural arrangement or configuration of the atoms. The term “tautomer,” as used herein, refers to one of two or more structural isomers which exist in equilibrium and which are readily converted from one isomeric form to another. It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers (stereoisomers) as well as enantiomeric and diastereomeric mixtures of the present compounds are within the scope of the disclosure.

The compounds described herein may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds described herein, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a [n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R³ substituents are present, each R³ substituent may be distinguished as R^{3A}, R^{3B}, wherein each of R^{3A}, R^{3B}, is defined within the scope of the definition of R³ and optionally differently.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “bond” or “bonded” refers to direct bonds, such as covalent bonds (e.g., direct or a linking group), or indirect bonds, such as non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions, and the like).

The terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N-hydroxysuccinimide, or —maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al, Modification of Proteins, Advances in Chemistry Series, Vol. 198, American Chemical Society, Washington, D.C. , 1982. In embodiments, the first bioconjugate reactive group (e.g., unnatural amino acid side chain) is covalently attached to the second bioconjugate reactive group (e.g., a hydroxyl group).

The term “electron-withdrawing group” refers to a chemical moiety or substituent that removes electron density from a conjugated pi-electron system, thereby making the pi electron system less electrophilic.

The term “electron-donating group” refers to a chemical moiety or substituent that can donate electron density into a conjugated pi-electron system, thereby making the pi electron system more nucleophilic.

The terms “bind” and “bound” as used herein is used in accordance with its plain and ordinary meaning and refers to the association between atoms or molecules. The association can be direct or indirect. For example, bound atoms or molecules may be bound, e.g., by covalent bond, linker (e.g. a first linker or second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g., a single-domain antibody or a recombinant protein as described herein, i.e., comprising an unnatural amino acid side chain that is capable of binding to an amino acid residue on a different protein) that is able to measurably bind to a target. In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

### Proteins

Provided herein is a method of enhancing the bioreactivity and/or binding efficacy of a protein comprising: (i) mutating a first amino acid to an unnatural amino acid, and (ii) mutating a second amino acid proximal to the first amino acid to arginine; wherein the unnatural amino acid comprises a side chain of Formula (I) as described herein; thereby enhancing the bioreactivity and/or binding efficacy of the protein. In embodiments, the method of enhancing the bioreactivity and/or binding efficacy of a protein comprises: (i) mutating a first amino acid to an unnatural amino acid, and (ii) mutating a second amino acid proximal to the first amino acid to arginine; wherein the first amino acid is Arg, Ala, Ile, Leu, Met, Val, Phe, Trp, Tyr, Asn, Cys, Gln, Ser, Thr, Asp, Glu, His, Lys, Gly, or Pro; wherein the second amino acid is Ala, Ile, Leu, Met, Val, Phe, Trp, Tyr, Asn, Cys, Gln, Ser, Thr, Asp, Glu, His, Lys, Gly, or Pro; and wherein the unnatural amino acid comprises a side chain of Formula (I) as described herein; thereby enhancing the bioreactivity and/or binding efficacy of the protein. Mutating a second amino acid to arginine results in the “non-naturally occurring arginine.” In embodiments, the unnatural amino acid comprises a side chain of Formula (II) as described herein. In embodiments, the unnatural amino acid comprises a side chain of Formula (III) as described herein. In embodiments, the unnatural amino acid comprises a side chain of Formula (IV) as described herein. In embodiments, the unnatural amino acid comprises a side chain of Formula (V) as described herein. In embodiments, the unnatural amino acid comprises a side chain of Formula (VI) as described herein. In embodiments, the unnatural amino acid comprises a side chain of Formula (VII) as described herein. In embodiments, the unnatural amino acid comprises a side chain of Formula (VIII) as described herein. In embodiments, the unnatural amino acid comprises a side chain of Formula (IX) as described herein. In embodiments, the unnatural amino acid comprises a side chain of Formula (X) as described herein. In embodiments, the unnatural amino acid comprises a side chain of Formula (FSY) as described herein. In embodiments, the unnatural amino acid comprises a side chain of Formula (mFSY) as described herein. In embodiments, the unnatural amino acid comprises a side chain of Formula (FFY) as described herein. In embodiments, the unnatural amino acid comprises a side chain of Formula (FSK) as described herein. In embodiments, the unnatural amino acid comprises a side chain of Formula (mFSK) as described herein. In embodiments, the unnatural amino acid comprises a side chain of Formula (NHSF) as described herein. In embodiments, the protein is an antibody or an antibody variant. In embodiments, the antibody variant is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the method further comprises contacting the protein with a target protein, thereby covalently bonding the protein to the target protein.

Provided herein is a method of enhancing the bioreactivity and/or binding efficacy of a protein comprising an unnatural amino acid, the method comprising mutating an amino acid proximal to the unnatural amino acid to arginine; wherein the unnatural amino acid comprises a side chain of Formula (I) as described herein; thereby enhancing the bioreactivity and/or binding efficacy of the protein. In embodiments, the method of enhancing the bioreactivity and/or binding efficacy of a protein comprising an unnatural amino acid comprises mutating an amino acid proximal to the unnatural amino acid to arginine; wherein the amino acid is Ala, Ile, Leu, Met, Val, Phe, Trp, Tyr, Asn, Cys, Gln, Ser, Thr, Asp, Glu, His, Lys, Gly, or Pro; and wherein the unnatural amino acid comprises a side chain of Formula (I) as described herein; thereby enhancing the bioreactivity and/or binding efficacy of the protein. Mutating the amino acid to arginine results in the “non-naturally occurring arginine.” In embodiments, the unnatural amino acid comprises a side chain of Formula (II) as described herein. In embodiments, the unnatural amino acid comprises a side chain of Formula (III) as described herein. In embodiments, the unnatural amino acid comprises a side chain of Formula (IV) as described herein. In embodiments, the unnatural amino acid comprises a side chain of Formula (V) as described herein. In embodiments, the unnatural amino acid comprises a side chain of Formula (VI) as described herein. In embodiments, the unnatural amino acid comprises a side chain of Formula (VII) as described herein. In embodiments, the unnatural amino acid comprises a side chain of Formula (VIII) as described herein. In embodiments, the unnatural amino acid comprises a side chain of Formula (IX) as described herein. In embodiments, the unnatural amino acid comprises a side chain of Formula (X) as described herein. In embodiments, the unnatural amino acid comprises a side chain of Formula (FSY) as described herein. In embodiments, the unnatural amino acid comprises a side chain of Formula (mFSY) as described herein. In embodiments, the unnatural amino acid comprises a side chain of Formula (FFY) as described herein. In embodiments, the unnatural amino acid comprises a side chain of Formula (FSK) as described herein. In embodiments, the unnatural amino acid comprises a side chain of Formula (mFSK) as described herein. In embodiments, the unnatural amino acid comprises a side chain of Formula (NHSF) as described herein. In embodiments, the protein is an antibody or an antibody variant. In embodiments, the antibody variant is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the method further comprises contacting the protein with a target protein, thereby covalently bonding the protein to the target protein.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (I):

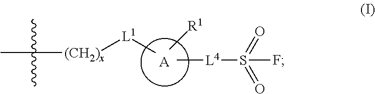

wherein: ring A is phenyl, a 5-membered cycloalkyl, a 5-membered heterocycloalkyl, or a 5-membered heteroaryl; L4 is a bond or —O—; x is an integer from 0 to 8; L1 is a bond, substituted or unsubstituted alkylene, or substituted or unsubstituted heteroalkylene; R1 is hydrogen, halogen, —CX¹₃, —CHX¹₂, —CH₂X1, —OCX¹₃, —OCH₂X1, —OCHX¹₂, —CN, —SOMIR^{1A}, —SOᵥ₁NR^{1A}R^{1B}, —NHC(O)NR^{1A}R^{1B}, —N(O)ₘ₁, —NR^{1A}R^{1B}, —C(O)R14, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR 1B substituted or unsubstituted alkyl, or substituted or unsubstituted heteroalkyl; X1 is independently-F, —C₁, -Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2. In embodiments, the protein further comprises a naturally occurring arginine. In embodiments, the protein further comprises a naturally occurring arginine that is not proximal to the —S(O₂) F group in the unnatural amino acid side chain. R1 is ortho, meta, or para to —S(O₂)F. In embodiments, R¹ is meta or ortho to —S(O₂)F. In embodiments, R¹ is meta to —S(O₂)F. In embodiments, R¹ is ortho to —S(O₂)F. In embodiments, R¹ is hydrogen, halogen, —CX¹₃, —CHX¹₂, —CH₂X1, —OCX¹₃, —OCH₂X1, —OCHX¹₂, —CN, —SOMIR^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, unsubstituted C₁₋₈ alkyl, or unsubstituted 2 to 8 membered heteroalkyl; R^{1A} is hydrogen, unsubstituted C₁₋₄ alkyl, or unsubstituted 2 to 4 membered heteroalkyl; and R^{1B} is hydrogen, unsubstituted C₁₋₄ alkyl, or unsubstituted 2 to 4 membered heteroalkyl. In embodiments, R¹ is hydrogen or halogen.

Provided herein are proteins comprising: (i) an unnatural amino acid, and (ii) a naturally-occurring arginine; wherein: (a) the naturally occurring arginine is proximal to the —S(O₂) F group in the unnatural amino acid side chain; and (b) the unnatural amino comprises a side chain of Formula (I):

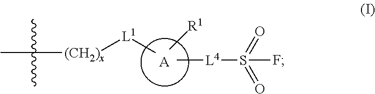

wherein: ring A is phenyl, a 5-membered cycloalkyl, a 5-membered heterocycloalkyl, or a 5-membered heteroaryl; L4 is a bond or —O—; x is an integer from 0 to 8; L1 is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹₂, —CN, —SOnIR^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)-OR^{1A}, —C(O)NR^{1A}R^{1B}, —OR14, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2; provided that when A is phenyl, then the protein is not epidermal growth factor receptor, protein tyrosine phosphatase 1B, P-selectin glycoprotein ligand 1 (PGSL-1), complement component 5a receptor (C5aR), chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377. In embodiments, when A is a 5-membered cycloalkyl, a 5-membered heterocycloalkyl, or a 5-membered heteroaryl, then the protein is not enhanced green fluorescent protein, a maltose binding protein, or protein Z. In embodiments, the protein does not comprise a non-naturally occurring arginine. In embodiments, R¹ is para, ortho, meta to —S(O₂)F. In embodiments, R¹ is meta to —S(O₂)F. In embodiments, R¹ is ortho to —S(O₂)F. In embodiments, R¹ is hydrogen, halogen, —CX¹₃, —CHX¹₂, —CH₂X¹, —OCX¹₃, —OCH₂X¹, —OCHX¹₂, —CN, —SOn1R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, unsubstituted C₁₋₈ alkyl, or unsubstituted 2 to 8 membered heteroalkyl; RIA is hydrogen, unsubstituted C₁₋₄ alkyl, or unsubstituted 2 to 4 membered heteroalkyl; and R^{1B} is hydrogen, unsubstituted C₁₋₄ alkyl, or unsubstituted 2 to 4 membered heteroalkyl. In embodiments, R¹ is hydrogen or halogen.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (II):

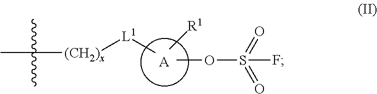

wherein: ring A is phenyl a 5-membered cycloalkyl, a 5-membered heterocycloalkyl, or a 5-membered heteroaryl; x is an integer from 0 to 8; L1 is a bond, substituted or unsubstituted alkylene, or substituted or unsubstituted heteroalkylene; R1 is hydrogen, halogen, —CX¹₃, —CHX¹₂, —CH₂X¹, —OCX¹₃, —OCH₂X¹, —OCHX¹₂, —CN, —SOM1R14, —SOviNR^{1A}R^{1B}, −NHC(O)NR^{1A}R^{1B}, —N(O)ₘ₁, —NR^{1A}R^{1B}, —C(O)R^{1A}, —C(O)-OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2. In embodiments, the protein further comprises a naturally occurring arginine. In embodiments, the protein further comprises a naturally occurring arginine that is not proximal to the —S(O₂) F group in the unnatural amino acid side chain. R1 is ortho, meta, or para to —S(O₂)F. In embodiments, R¹ is meta or ortho to —S(O₂)F. In embodiments, R¹ is meta to —S(O₂)F. In embodiments, R¹ is ortho to —S(O₂)F. In embodiments, R1 is hydrogen, halogen, —CX¹₃, —CHX¹₂, —CH₂X¹, —OCX¹₃, —OCH₂X¹, —OCHX¹₂, —CN, —SOM1R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, unsubstituted C₁₋₈ alkyl, or unsubstituted 2 to 8 membered heteroalkyl; R^{1A} is hydrogen, unsubstituted C₁₋₄ alkyl, or unsubstituted 2 to 4 membered heteroalkyl; and R^{1B} is hydrogen, unsubstituted C₁₋₄ alkyl, or unsubstituted 2 to 4 membered heteroalkyl. In embodiments, R¹ is hydrogen or halogen.

Provided herein are proteins comprising: (i) an unnatural amino acid, and (ii) a naturally-occurring arginine; wherein: (a) the naturally occurring arginine is proximal to the —S(O₂)F group in the unnatural amino acid side chain; and (b) the unnatural amino comprises a side chain of Formula (II):

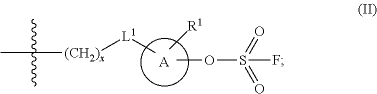

wherein: ring A is phenyl, a 5-membered cycloalkyl, a 5-membered heterocycloalkyl, or a 5-membered heteroaryl; x is an integer from 0 to 8; L′ is a bond, substituted or unsubstituted alkylene, or substituted or unsubstituted heteroalkylene; R1 is hydrogen, halogen, —CX¹₃, —CHX¹₂, —CH₂X¹, —OCX¹₃, —OCH₂X¹, —OCHX¹₂, —CN, —SOₘ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR14, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2; provided that when A is phenyl, then the protein is not epidermal growth factor receptor, protein tyrosine phosphatase 1B, P-selectin glycoprotein ligand 1 (PGSL-1), complement component 5a receptor (C5aR), chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377. In embodiments, when A is a 5-membered cycloalkyl, a 5-membered heterocycloalkyl, or a 5-membered heteroaryl, then the protein is not enhanced green fluorescent protein, a maltose binding protein, or protein Z. In embodiments, the protein does not comprise a non-naturally occurring arginine. In embodiments, R¹ is para, ortho, meta to —S(O₂)F. In embodiments, R¹ is meta to —S(O₂)F. In embodiments, R¹ is ortho to —S(O₂)F. In embodiments, R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOV1NR^{1A}R^{1B}, —NHC(O)NR^{1A}R^{1B}, —N(O)ₘ₁, —NR^{1A}R^{1B}, —C(O)R^{1A}, —C(O)-OR^{1A}, —C(O)NR^{1A}R^{1B}, —OR^{1A}, —NR^{1A}SO₂R^{1B}, —NR 1AC(O)R^{1B}, —NR^{1A}C(O)OR^{1B}, —NR 1AOR^{1B}, unsubstituted C₁₋₈ alkyl, or unsubstituted 2 to 8 membered heteroalkyl; R^{1A} is hydrogen, unsubstituted C₁₋₄ alkyl, or unsubstituted 2 to 4 membered heteroalkyl; and R^{1B} is hydrogen, unsubstituted C₁₋₄ alkyl, or unsubstituted 2 to 4 membered heteroalkyl. In embodiments, R¹ is hydrogen or halogen.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (III):

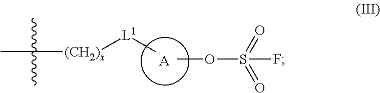

wherein ring A is phenyl, a 5-membered cycloalkyl, a 5-membered heterocycloalkyl, or a 5-membered heteroaryl; x is an integer from 0 to 8; L1 is a bond, substituted or unsubstituted alkylene, or substituted or unsubstituted heteroalkylene. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally-occurring arginine; wherein: (a) the naturally occurring arginine is proximal to the —S(O₂) F group in the unnatural amino acid side chain; and (b) the unnatural amino comprises a side chain of Formula (III):

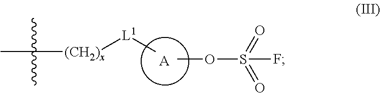

wherein ring A is phenyl, a 5-membered cycloalkyl, a 5-membered heterocycloalkyl, or a 5-membered heteroaryl; x is an integer from 0 to 8; L1 is a bond, substituted or unsubstituted alkylene, or substituted or unsubstituted heteroalkylene; provided that when A is phenyl, then the protein is not epidermal growth factor receptor, protein tyrosine phosphatase 1B, P-selectin glycoprotein ligand 1 (PGSL-1), complement component 5a receptor (C5aR), chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377. In embodiments, when A is a 5-membered cycloalkyl, a 5-membered heterocycloalkyl, or a 5-membered heteroaryl, then the protein is not enhanced green fluorescent protein, a maltose binding protein, or protein Z. In embodiments, the protein does not comprise a non-naturally occurring arginine.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (FSY):

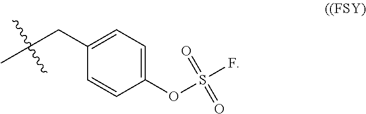 [SMILES: *Cc1ccc(OS(=O)(=O)F)cc1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂)F group in the unnatural amino acid side chain; and (b) the unnatural amino comprises a side chain of Formula (FSY):

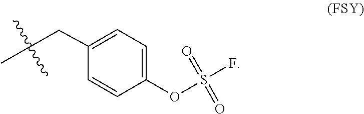 [SMILES: *Cc1ccc(OS(=O)(=O)F)cc1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not epidermal growth factor receptor, protein tyrosine phosphatase 1B, P-selectin glycoprotein ligand 1, complement component 5a receptor, chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (mFSY):

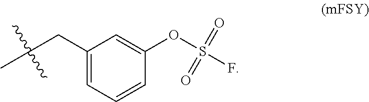 [SMILES: *Cc1cccc(OS(=O)(=O)F)c1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂) F group in the unnatural amino acid side chain; and (b) the unnatural amino comprises a side chain of Formula (mFSY):

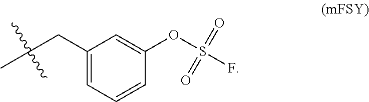 [SMILES: *Cc1cccc(OS(=O)(=O)F)c1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not epidermal growth factor receptor, protein tyrosine phosphatase 1B, P-selectin glycoprotein ligand 1, complement component 5a receptor, chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (FFY):

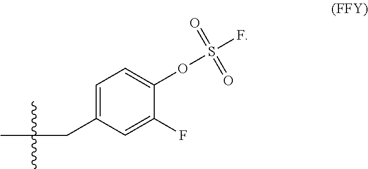 [SMILES: *Cc1ccc(OS(=O)(=O)F)c(F)c1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂) F group in the unnatural amino acid side chain; and (b) the unnatural amino comprises a side chain of Formula (FFY):

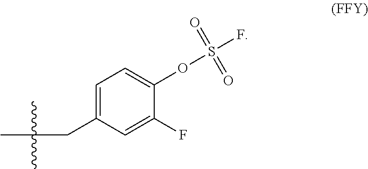 [SMILES: *Cc1ccc(OS(=O)(=O)F)c(F)c1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not epidermal growth factor receptor, protein tyrosine phosphatase 1B, P-selectin glycoprotein ligand 1, complement component 5a receptor, chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (FSK):

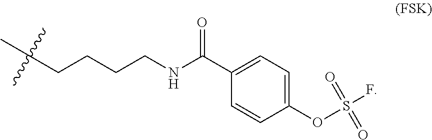 [SMILES: *CCCCNC(=O)c1ccc(OS(=O)(=O)F)cc1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂)F group in the unnatural amino acid side chain; and (b) the unnatural amino comprises a side chain of Formula (FSK):

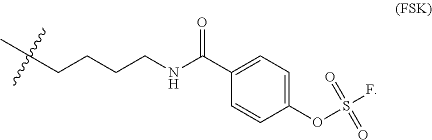 [SMILES: *CCCCNC(=O)c1ccc(OS(=O)(=O)F)cc1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not epidermal growth factor receptor, protein tyrosine phosphatase 1B, P-selectin glycoprotein ligand 1, complement component 5a receptor, chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (mFSK):

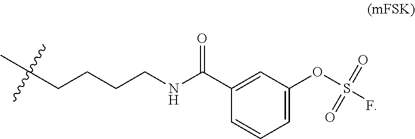 [SMILES: *CCCCNC(=O)c1cccc(OS(=O)(=O)F)c1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S (02) F group in the unnatural amino acid side chain; and (b) the unnatural amino comprises a side chain of Formula (mFSK):

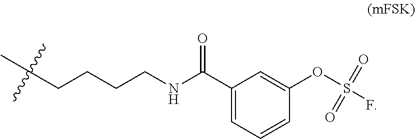 [SMILES: *CCCCNC(=O)c1cccc(OS(=O)(=O)F)c1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not epidermal growth factor receptor, protein tyrosine phosphatase 1B, P-selectin glycoprotein ligand 1, complement component 5a receptor, chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (IV):

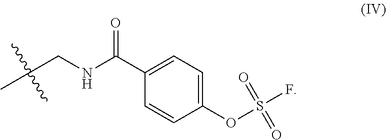 [SMILES: *CNC(=O)c1ccc(OS(=O)(=O)F)cc1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S (02) F group in the unnatural amino acid side chain; and (b) the unnatural amino comprises a side chain of Formula (IV):

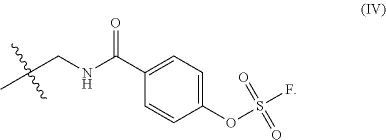 [SMILES: *CNC(=O)c1ccc(OS(=O)(=O)F)cc1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not epidermal growth factor receptor, protein tyrosine phosphatase 1B, P-selectin glycoprotein ligand 1, complement component 5a receptor, chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (V):

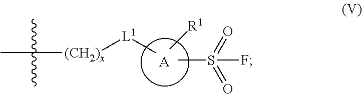

wherein: ring A is phenyl, a 5-membered cycloalkyl, a 5-membered heterocycloalkyl, or a 5-membered heteroaryl; x is an integer from 0 to 8; L1 is a bond, substituted or unsubstituted alkylene, or substituted or unsubstituted heteroalkylene; R1 is hydrogen, halogen, —CX¹₃, —CHX¹₂, —CH₂X¹, —OCX¹₃, —OCH₂X¹, —OCHX¹₂, —CN, —SOMIR^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2. In embodiments, the protein further comprises a naturally occurring arginine. In embodiments, the protein further comprises a naturally occurring arginine that is not proximal to the —S(O₂) F group in the unnatural amino acid side chain. R¹ is ortho, meta, or para to —S(O₂)F. In embodiments, R¹ is meta or ortho to —S(O₂)F. In embodiments, R¹ is meta to —S(O₂)F. In embodiments, R¹ is ortho to —S(O₂)F. In embodiments, R¹ is hydrogen, halogen, —CX¹₃, —CHX¹₂, —CH₂X¹, —OCX¹₃, —OCH₂X¹, —OCHX¹₂, —CN, —SOₘ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, unsubstituted C₁₋₈ alkyl, or unsubstituted 2 to 8 membered heteroalkyl; R^{1A} is hydrogen, unsubstituted C₁₋₄ alkyl, or unsubstituted 2 to 4 membered heteroalkyl; and R^{1B} is hydrogen, unsubstituted C₁₋₄ alkyl, or unsubstituted 2 to 4 membered heteroalkyl. In embodiments, R¹ is hydrogen or halogen.

Provided herein are proteins comprising: (i) an unnatural amino acid, and (ii) a naturally occurring arginine; wherein: (a) the naturally occurring arginine is proximal to the —S(O₂)F group in the unnatural amino acid side chain; and (b) the unnatural amino comprises a side chain of Formula (V):

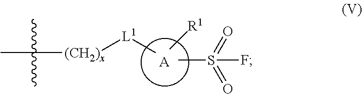

wherein: ring A is phenyl, a 5-membered cycloalkyl, a 5-membered heterocycloalkyl, or a 5-membered heteroaryl; x is an integer from 0 to 8; L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹₂, —CN, —SOniR^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2 provided that when A is phenyl, then the protein is not epidermal growth factor receptor, protein tyrosine phosphatase 1B, P-selectin glycoprotein ligand 1 (PGSL-1), complement component 5a receptor (C5aR), chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377. In embodiments, when A is a 5-membered cycloalkyl, a 5-membered heterocycloalkyl, or a 5-membered heteroaryl, then the protein is not enhanced green fluorescent protein, a maltose binding protein, or protein Z. In embodiments, the protein does not comprise a non-naturally occurring arginine. In embodiments, R¹ is para, ortho, meta to —S(O₂)F. In embodiments, R¹ is meta to —S(O₂)F. In embodiments, R¹ is ortho to —S(O₂)F. In embodiments, R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₂NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, unsubstituted C₁₋₈ alkyl, or unsubstituted 2 to 8 membered heteroalkyl; R^{1A} is hydrogen, unsubstituted C₁₋₄ alkyl, or unsubstituted 2 to 4 membered heteroalkyl; and R^{1B} is hydrogen, unsubstituted C₁₋₄ alkyl, or unsubstituted 2 to 4 membered heteroalkyl. In embodiments, R¹ is hydrogen or halogen.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (VI):

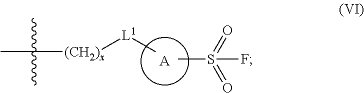

wherein ring A is phenyl, a 5-membered cycloalkyl, a 5-membered heterocycloalkyl, or a 5-membered heteroaryl; x is an integer from 0 to 8; L¹ is a bond, substituted or unsubstituted alkylene, or substituted or unsubstituted heteroalkylene. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂) F group in the unnatural amino acid side chain; and (b) the unnatural amino comprises a side chain of Formula (VI):

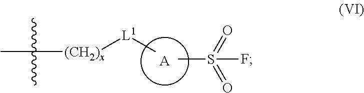

wherein ring A is phenyl, a 5-membered cycloalkyl, a 5-membered heterocycloalkyl, or a 5-membered heteroaryl; x is an integer from 0 to 8; L¹ is a bond, substituted or unsubstituted alkylene, or substituted or unsubstituted heteroalkylene provided that when A is phenyl, then the protein is not epidermal growth factor receptor, protein tyrosine phosphatase 1B, P-selectin glycoprotein ligand 1 (PGSL-1), complement component 5a receptor (C5aR), chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377. In embodiments, when A is a 5-membered cycloalkyl, a 5-membered heterocycloalkyl, or a 5-membered heteroaryl, then the protein is not enhanced green fluorescent protein, a maltose binding protein, or protein Z. In embodiments, the protein does not comprise a non-naturally occurring arginine.

In embodiments of the compounds described herein, ring A is phenyl, a 5-membered cycloalkyl, a 5-membered heterocycloalkyl, or a 5-membered heteroaryl. In embodiments, ring A is phenyl. In embodiments of the compounds described herein, ring A is a 5-membered cycloalkyl, a 5-membered heterocycloalkyl, or a 5-membered heteroaryl. In embodiments, ring A is a 5-membered cycloalkyl. In embodiments, ring A is a 5-membered cycloalkyl having no C═C double bonds. In embodiments, ring A is a 5-membered cycloalkyl having one C═C double bond. In embodiments, ring A is a 5-membered cycloalkyl having two C═C double bonds. In embodiments, ring A is a 5-membered heterocycloalkyl. In embodiments, ring A is a 5-membered heterocycloalkyl having no double bonds. In embodiments, ring A is a 5-membered heterocycloalkyl having one double bond.

In embodiments, ring A is a 5-membered heteroaryl. In embodiments, ring A is a 5-membered heteroaryl containing 1 to 4 heteroatoms selected from the group consisting of oxygen, nitrogen, and sulfur. In embodiments, ring A is a 5-membered heteroaryl containing 1 to 3 heteroatoms selected from the group consisting of oxygen, nitrogen, and sulfur. In embodiments, ring A is a 5-membered heteroaryl containing 1 or 2 heteroatoms selected from the group consisting of oxygen, nitrogen, and sulfur. In embodiments, ring A is a 5-membered heteroaryl containing 1 heteroatom selected from the group consisting of oxygen, nitrogen, and sulfur. In embodiments, ring A is a 5-membered heteroaryl containing 2 heteroatoms selected from the group consisting of oxygen, nitrogen, and sulfur. In embodiments, ring A is a 5-membered heteroaryl containing 3 heteroatoms selected from the group consisting of oxygen, nitrogen, and sulfur. In embodiments, ring A is pyrrole, pyrazole, imidazole, triazole, furan, thiophene, phosphole, oxazole, isoxazole, thiazole, or isothiazole. In embodiments, ring A is pyrrole. In embodiments, ring A is pyrazole. In embodiments, ring A is imidazole. In embodiments, ring A is triazole. In embodiments, ring A is furan. In embodiments, ring A is thiophene. In embodiments, ring A is phosphole. In embodiments, ring A is oxazole. In embodiments, ring A is isoxazole. In embodiments, ring A is thiazole. In embodiments, ring A is isothiazole. In embodiments, L¹ is attached to a heteroatom in the 5-membered heteroaryl. In embodiments, L¹ is attached to a carbon atom in the 5-membered heteroaryl. In embodiments, the —S(O₂) F moiety is attached to a heteroatom in the 5-membered heteroaryl. In embodiments, the —S(O₂) F moiety is attached to a carbon atom in the 5-membered heteroaryl. In embodiments, L′ is attached to a carbon atom in the 5-membered heteroaryl and the —S(O₂) F moiety is attached to a carbon atom in the 5-membered heteroaryl. In embodiments, L¹ is attached to a heteroatom in the 5-membered heteroaryl and the —S(O₂) F moiety is attached to a carbon atom in the 5-membered heteroaryl. In embodiments, L¹ is attached to a carbon atom in the 5-membered heteroaryl and the —S(O₂) F moiety is attached to a heteroatom in the 5-membered heteroaryl. In embodiments, L¹ is attached to a heteroatom in the 5-membered heteroaryl, and the —S(O₂) F moiety is attached to a heteroatom in the 5-membered heteroaryl.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (NHSF):

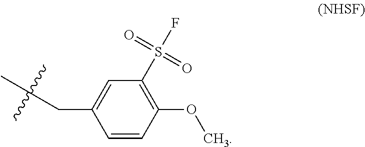 [SMILES: *Cc1ccc(OC)c(S(=O)(=O)F)c1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂) F group in the unnatural amino acid side chain; and (b) the unnatural amino comprises a side chain of Formula (NHSF):

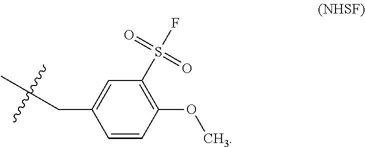 [SMILES: *Cc1ccc(OC)c(S(=O)(=O)F)c1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not epidermal growth factor receptor, protein tyrosine phosphatase 1B, P-selectin glycoprotein ligand 1, complement component 5a receptor, chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (VII):

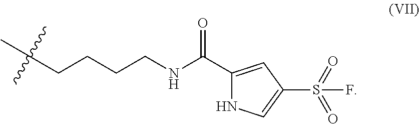 [SMILES: *CCCCNC(=O)c1cc(S(=O)(=O)F)c[nH]1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂) F group in the unnatural amino acid side chain; and (b) the unnatural amino comprises a side chain of Formula (VII):

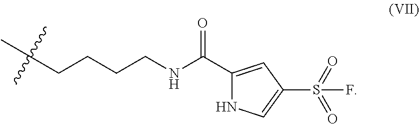 [SMILES: *CCCCNC(=O)c1cc(S(=O)(=O)F)c[nH]1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not enhanced green fluorescent protein, a maltose binding protein, or protein Z.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (VIII):

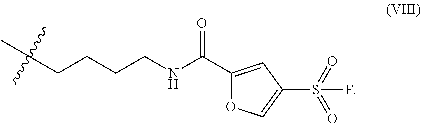 [SMILES: *CCCCNC(=O)c1cc(S(=O)(=O)F)co1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂) F group in the unnatural amino acid side chain; and (b) the unnatural amino comprises a side chain of Formula (VIII):

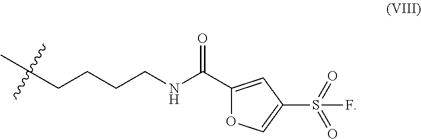 [SMILES: *CCCCNC(=O)c1cc(S(=O)(=O)F)co1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not enhanced green fluorescent protein, a maltose binding protein, or protein Z.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (IX):

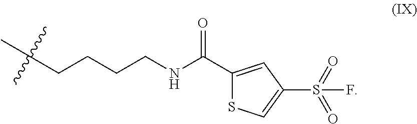 [SMILES: *CCCCNC(=O)c1cc(S(=O)(=O)F)cs1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂) F group in the unnatural amino acid side chain; and (b) the unnatural amino comprises a side chain of Formula (IX):

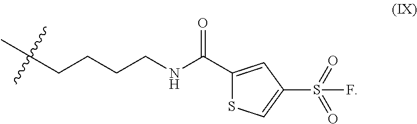 [SMILES: *CCCCNC(=O)c1cc(S(=O)(=O)F)cs1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not enhanced green fluorescent protein, a maltose binding protein, or protein Z.

Provided herein are proteins comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (X):

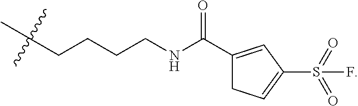 [SMILES: *CCCCNC(=O)C1=CC(S(=O)(=O)F)=CC1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein further comprises a naturally occurring arginine. In embodiments, the protein further comprises a naturally occurring arginine that is not proximal to the —S(O₂) F group in the unnatural amino acid side chain.

Provided herein are proteins comprising: (i) an unnatural amino acid, and (ii) a naturally occurring arginine; wherein: (a) the naturally occurring arginine is proximal to the —S(O₂) F group in the unnatural amino acid side chain; and (b) the unnatural amino comprises a side chain of Formula (X):

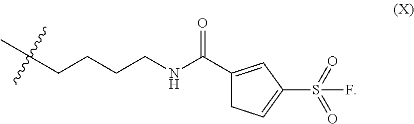 [SMILES: *CCCCNC(=O)C1=CC(S(=O)(=O)F)=CC1]

In embodiments, the protein is an antibody. In embodiments, the protein is a single-chain variable fragment, a single-domain antibody, an affibody, or an antigen-binding fragment. In embodiments, the protein is a single-chain variable fragment. In embodiments, the protein is a single-domain antibody. In embodiments, the protein is an affibody. In embodiments, the protein is an antigen-binding fragment. In embodiments, the protein does not comprise a non-naturally occurring arginine. In embodiments, the protein is not enhanced green fluorescent protein, a maltose binding protein, or protein Z.

In embodiments of the compounds described herein, the protein is an antibody, an antibody variant. In embodiments, the protein is an antibody. In embodiments, the protein is an antibody variant. In embodiments, the antibody variant is a variant as defined herein. In embodiments, the antibody variant is a single-chain variable fragment, a single-domain antibody, an affibody, or an antigen-binding fragment. In embodiments, the antibody variant is a single-chain variable fragment. In embodiments, the antibody variant is a single-domain antibody. In embodiments, the antibody variant is an affibody. In embodiments, the antibody variant is or an antigen-binding fragment. In embodiment, the unnatural amino acid and the arginine (non-naturally occurring or naturally occurring) are within a CDR region or a framework region of the antibody. In embodiment, the unnatural amino acid and the arginine (non-naturally occurring or naturally occurring) are within a CDR region of the antibody. In embodiment, the unnatural amino acid is within a framework region of the antibody and the arginine (non-naturally occurring or naturally occurring) are within a CDR region of the antibody. In embodiments, the unnatural amino acid and the arginine (non-naturally occurring or naturally occurring) are within the same CDR region of the antibody. In embodiments, the unnatural amino acid and the arginine (non-naturally occurring or naturally occurring) are within different CDR regions of the antibody. In embodiment, the unnatural amino acid is within a CDR region of the antibody and the arginine (non-naturally occurring or naturally occurring) is within a framework region of the antibody. In embodiment, the unnatural amino acid and the arginine (non-naturally occurring or naturally occurring) are within a CDR region or a framework region of the antibody variant. In embodiment, the unnatural amino acid and the arginine (non-naturally occurring or naturally occurring) are within a CDR region of the antibody variant. In embodiment, the unnatural amino acid is within a framework region of the antibody variant and the arginine (non-naturally occurring or naturally occurring) is within a CDR region of the antibody variant. In embodiments, the unnatural amino acid and the arginine (non-naturally occurring or naturally occurring) are within the same CDR region of the antibody variant. In embodiments, the unnatural amino acid and the arginine (non-naturally occurring or naturally occurring) are within different CDR regions of the antibody variant. In embodiment, the unnatural amino acid is within a CDR region of the antibody variant and the arginine (non-naturally occurring or naturally occurring) is within a framework region of the antibody variant.

In embodiments, the single-domain antibody has at least 90% sequence identity to the amino acid sequence of SEQ ID NO: 1. In embodiments, the single-domain antibody has at least 95% sequence identity to the amino acid sequence of SEQ ID NO:1. In embodiments, the single-domain antibody comprises the amino acid sequence of SEQ ID NO:1. In embodiments, the single-domain antibody is the amino acid sequence of SEQ ID NO:1. In embodiments, the unnatural amino acid is within a CDR region of SEQ ID NO:1, and the non-naturally occurring arginine is within a CDR region of SEQ ID NO:1. In embodiments, the unnatural amino acid is within a CDR region of SEQ ID NO:1, and the non-naturally occurring arginine is within a framework region of SEQ ID NO:1. In embodiments, the position corresponding to position 44 in SEQ ID NO: 1 is arginine and the position corresponding to position 109 in SEQ ID NO: 1 is an unnatural amino acid having an unnatural amino acid side chain described herein. Provided herein is a pharmaceutical composition comprising SEQ ID NO:1 (or any embodiment thereof) and a pharmaceutically acceptable carrier.

Provided herein is a method of covalently binding a single-domain antibody to epidermal growth factor receptor comprising contacting the single domain antibody with the epidermal growth factor receptor; wherein the single-domain antibody comprises SEQ ID NO:1 (or any embodiment thereof), thereby covalently binding the single-domain antibody to the epidermal growth factor receptor. Provided herein is a biomolecule conjugate comprising a single-domain antibody covalently bonded to epidermal growth factor receptor, wherein the single-domain antibody comprises SEQ ID NO:1 (or any embodiment thereof).

In embodiments, the single-domain antibody has at least 90% sequence identity to SEQ ID NO: 2, provided that the position corresponding to position 44 in SEQ ID NO:2 is arginine and the position corresponding to position 109 in SEQ ID NO:2 is FSY. In embodiments, the single-domain antibody has at least 95% sequence identity to SEQ ID NO:2, provided that the position corresponding to position 44 in SEQ ID NO:2 is arginine and the position corresponding to position 109 in SEQ ID NO:2 is FSY. In embodiments, the single-domain antibody comprises SEQ ID NO:2. Provided herein is a pharmaceutical composition comprising SEQ ID NO:2 (or any embodiment thereof) and a pharmaceutically acceptable carrier.

Provided herein is a method of covalently binding a single-domain antibody to epidermal growth factor receptor comprising contacting the single domain antibody with the epidermal growth factor receptor; wherein the single-domain antibody comprises SEQ ID NO:2 (or any embodiment thereof), thereby covalently binding the single-domain antibody to the epidermal growth factor receptor. Provided herein is a biomolecule conjugate comprising a single-domain antibody covalently bonded to epidermal growth factor receptor, wherein the single-domain antibody comprises SEQ ID NO:2 (or any embodiment thereof).

In embodiments, the single-domain antibody has at least 90% sequence identity to the amino acid sequence of SEQ ID NO:8. In embodiments, the single-domain antibody has at least 95% sequence identity to the amino acid sequence of SEQ ID NO:8. In embodiments, the single-domain antibody comprises the amino acid sequence of SEQ ID NO:8. In embodiments, the single-domain antibody is the amino acid sequence of SEQ ID NO:8. In embodiments, the unnatural amino acid is within a CDR region of SEQ ID NO:8, and the non-naturally occurring arginine is within a CDR region of SEQ ID NO:8. In embodiments, the unnatural amino acid is within a CDR region of SEQ ID NO:8, and the non-naturally occurring arginine is within a framework region of SEQ ID NO:8. In embodiments, the position corresponding to position 54 in SEQ ID NO:8 is an unnatural amino acid having an unnatural amino acid side chain described herein and the position corresponding to position 56 in SEQ ID NO:8 is arginine. Provided herein is a pharmaceutical composition comprising SEQ ID NO:8 (or any embodiment thereof) and a pharmaceutically acceptable carrier.

Provided herein is a method of covalently binding a single-domain antibody to human epidermal growth factor receptor-2 comprising contacting the single domain antibody with the human epidermal growth factor receptor-2; wherein the single-domain antibody comprises SEQ ID NO: 8 (or any embodiment thereof), thereby covalently binding the single-domain antibody to the human epidermal growth factor receptor-2. Provided herein is a biomolecule conjugate comprising a single-domain antibody covalently bonded to human epidermal growth factor receptor-2, wherein the single-domain antibody comprises SEQ ID NO:8 (or any embodiment thereof).

In embodiments, the single-domain antibody has at least 90% sequence identity to SEQ ID NO: 9, provided that the position corresponding to position 54 in SEQ ID NO:9 is FSY and the position corresponding to position 56 in SEQ ID NO:9 is arginine. In embodiments, the single-domain antibody has at least 95% sequence identity to SEQ ID NO:9, provided that the position corresponding to position 54 in SEQ ID NO:9 is FSY and the position corresponding to position 56 in SEQ ID NO:9 is arginine. In embodiments, the single-domain antibody comprises SEQ ID NO:9. Provided herein is a pharmaceutical composition comprising SEQ ID NO:9 (or any embodiment thereof) and a pharmaceutically acceptable carrier.

Provided herein is a method of covalently binding a single-domain antibody to human epidermal growth factor receptor-2 comprising contacting the single domain antibody with the human epidermal growth factor receptor-2; wherein the single-domain antibody comprises SEQ ID NO: 9 (or any embodiment thereof), thereby covalently binding the single-domain antibody to the human epidermal growth factor receptor-2. Provided herein is a biomolecule conjugate comprising a single-domain antibody covalently bonded to human epidermal growth factor receptor-2, wherein the single-domain antibody comprises SEQ ID NO:9 (or any embodiment thereof).

In embodiments, the affibody has at least 90% sequence identity to the amino acid sequence of SEQ ID NO:14. In embodiments, the affibody has at least 95% sequence identity to the amino acid sequence of SEQ ID NO:14. In embodiments, the affibody comprises the amino acid sequence of SEQ ID NO: 14. In embodiments, the affibody is the amino acid sequence of SEQ ID NO:14. In embodiments, the position corresponding to position 36 in SEQ ID NO: 14 is an unnatural amino acid having an unnatural amino acid side chain described herein and the position corresponding to position 32 in SEQ ID NO:14 is arginine. Provided herein is a pharmaceutical composition comprising SEQ ID NO:14 (or any embodiment thereof) and a pharmaceutically acceptable carrier.

Provided herein is a method of covalently binding an affibody to protein Z comprising contacting the affibody with the protein Z; wherein the affibody comprises SEQ ID NO: 14 (or any embodiment thereof), thereby covalently binding the affibody to the protein Z. Provided herein is a biomolecule conjugate comprising an affibody covalently bonded to protein Z, wherein the affibody comprises SEQ ID NO: 14 (or any embodiment thereof).

In embodiments, the affibody has at least 90% sequence identity to SEQ ID NO:15, provided that the position corresponding to position 36 in SEQ ID NO:15 is FSY and the position corresponding to position 32 in SEQ ID NO:15 is arginine. In embodiments, the affibody has at least 95% sequence identity to SEQ ID NO:15, provided that the position corresponding to position 36 in SEQ ID NO: 15 is FSY and the position corresponding to position 32 in SEQ ID NO: 15 is arginine. In embodiments, the affibody comprises the amino acid sequence of SEQ ID NO: 15. In embodiments, the affibody is the amino acid sequence of SEQ ID NO:15. Provided herein is a pharmaceutical composition comprising SEQ ID NO:15 (or any embodiment thereof) and a pharmaceutically acceptable carrier.

Provided herein is a method of covalently binding an affibody to protein Z comprising contacting the affibody with the protein Z; wherein the affibody comprises SEQ ID NO: 15 (or any embodiment thereof), thereby covalently binding the affibody to the protein Z. Provided herein is a biomolecule conjugate comprising an affibody covalently bonded to protein Z, wherein the affibody comprises SEQ ID NO: 15 (or any embodiment thereof).

In embodiments of the compounds described herein, the protein is a receptor protein. In embodiments, the receptor protein is a programmed death-ligand 1 (PD-L1) receptor, a programmed cell death protein 1 (PD-1) receptor, a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SIP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, a vasopressin receptor, or a combination of two or more thereof. In embodiments, the receptor protein is an integrin. In embodiments, the receptor protein is a somatostatin receptor. In embodiments, the receptor protein is a gonadotropin-releasing hormone receptor. In embodiments, the receptor protein is a bombesin receptor. In embodiments, the receptor protein is a vasoactive intestinal peptide receptor. In embodiments, the receptor protein is a neurotensin receptor. In embodiments, the receptor protein is a cholecystokinin 2 receptor. In embodiments, the receptor protein is a melanocortin receptor. In embodiments, the receptor protein is a ghrelin receptor.

In embodiments, the receptor protein is a PD-L1 receptor or a PD-1 receptor. In embodiments, the receptor protein is a PD-L1 receptor. In embodiments, the receptor protein is a PD-1 receptor.

In embodiments, the receptor protein is a receptor expressed on a cancer cell. In embodiments, the receptor protein is a receptor overexpressed on a cancer cell relative to a control.

In embodiments, the receptor protein is a G protein-coupled receptor. In embodiments, the receptor protein is a receptor tyrosine kinase. In embodiments, the receptor protein is a an ErbB receptor. In embodiments, the receptor protein is an epidermal growth factor receptor (EGFR). In embodiments, the receptor protein is epidermal growth factor receptor 1 (HER1). In embodiments, the receptor protein is epidermal growth factor receptor 2 (HER2). In embodiments, the receptor protein is epidermal growth factor receptor 3 (HER3). In embodiments, the receptor protein is epidermal growth factor receptor 4 (HER4).

In embodiments, the protein is a cell surface receptor. In embodiments, the cell surface receptor is in the extracellular domain, the transmembrane domain, or the intracellular domain. In embodiments, the protein is a cytosolic protein. In embodiments, the protein is a transcriptional factor. In embodiments, the protein is a an enzyme.

In embodiments, the protein described herein further comprises a detectable agent or a therapeutic agent. In embodiments, the protein further comprises a detectable agent and a therapeutic agent. In embodiments, the protein further comprises a detectable agent. In embodiments, the detectable agent is a radioisotope. In embodiments, the protein further comprises a therapeutic agent.

In embodiments, the detectable label is a detectable label that can be used in medical imaging. In embodiments, the detectable label is a label that can be used for radiography, magnetic resonance imaging, nuclear medicine, ultrasound elastography, photoacoustic imaging, tomography, echocardiography, functional near-infrared spectroscopy, magnetic particle imaging. In embodiments, the detectable label is a label that can be use for tomography. In embodiments, the detectable label is a label that can be used for positron emission tomography.

A “detectable agent” or “detectable moiety” is a composition detectable by appropriate means such as spectroscopic, photochemical, biochemical, immunochemical, chemical, magnetic resonance imaging, or other physical means. In embodiments, the proteins described herein are bonded to a detectable agent. In embodiments, the fusion proteins described herein are bonded to a detectable agent. In embodiments, an antibody or antibody variant is bonded to a detectable agent. In embodiments, a nanobody is bonded to a detectable agent. In embodiments, the bond is noncovalent or covalent. In embodiments, the bond is covalent. In embodiments, the protein is covalently bonded to a detectable agent. In embodiments, the fusion protein is covalently bonded to a detectable agent. In embodiments, the antibody or antibody variant is covalently bonded to a detectable agent. In embodiments, a nanobody is covalently bonded to a detectable agent. In embodiments when the protein or fusion protein is covalently bonded to a detectable agent, the covalent bond is between the detectable agent and a naturally-occurring amino acid in the protein or fusion protein. In embodiments when the nanobody is covalently bonded to a detectable agent, the covalent bond is between the detectable agent and a naturally-occurring amino acid in the nanobody. Methods for covalently bonding detectable agents to proteins are well-known in the art. Detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷ As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹ Ag, ¹¹¹ In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴-¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸ Au, ¹⁹⁹ Au, ²¹¹ At, ²¹¹Pb, ²¹²Bi, ²¹²Pb, ²¹³Bi, ²²³Ra, ²²⁵ Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide nanoparticles, USPIO nanoparticle aggregates, superparamagnetic iron oxide (“SPIO”) nanoparticles, SPIO nanoparticle aggregates, monocrystalline iron oxide nanoparticles, monochrystalline iron oxide, nanoparticle contrast agents, liposomes or other delivery vehicles containing Gadolinium chelate molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles, iodinated contrast agents, barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition. In embodiments, paramagnetic ions that may be used as imaging agents in accordance with the embodiments of the disclosure include, e.g., ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

A “radioisotope” that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷ As, ⁸⁶Y, ⁹⁰Y. ⁸⁹Sr, ⁸⁹Zr, ⁹⁴Tc, ⁹⁴Tc, ⁹⁹mTc, ⁹⁹Mo, ¹⁰⁵Pd, ¹⁰⁵ Rh, ¹¹¹ Ag, ¹¹¹ In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴-¹⁵⁸¹Gd, ¹⁶¹Tb, ¹⁶⁶Dy, ¹⁶⁶Ho, ¹⁶⁹ Er, ¹⁷⁵Lu, ¹⁷⁷Lu, ¹⁸⁶Re, ¹⁸⁸Re, ¹⁸⁹Re, ¹⁹⁴Ir, ¹⁹⁸ Au, ¹⁹⁹ Au, ²¹¹ At, ²¹¹Pb, ²¹²Bi, ²¹²Pb, ²¹³Bi, ²²³Ra and ²²⁵ Ac. In embodiments, the proteins described herein are bonded to a radioisotope. In embodiments, an antibody or antibody variant is bonded to a radioisotope. In embodiments, a nanobody is bonded to a radioisotope. In embodiments, the bond is noncovalent or covalent. In embodiments, the bond is covalent. In embodiments, the protein is covalently bonded to a radioisotope. In embodiments, the antibody or antibody variant is covalently bonded to a radioisotope. In embodiments, a nanobody is covalently bonded to a radioisotope. In embodiments when the nanobody is covalently bonded to a radioisotope, the covalent bond is between the radioisotope and an unnatural amino acid in the nanobody. Methods for covalently bonding radioisotopes to proteins are well-known in the art. In embodiments, the radioisotope is ¹²³I, ¹²⁴I, ¹²⁵I, or ¹³¹I. In embodiments, the radioisotope is ¹²³I. In embodiments, the radioisotope is ¹²⁴I. In embodiments, the radioisotope is ¹²⁵I. In embodiments, the radioisotope is ¹³¹I. In embodiments, the radioisotope is a positron-emitting radioisotope. In embodiments, the positron-emitting radioisotope is ¹¹C, ¹³N, ¹⁵O, ¹⁸F, ⁶⁴Cu, ⁶⁸Ga, ⁷⁸Br, ⁸²Rb, ⁸⁶Y, ⁸⁹Zr, ⁹⁰Y, ²²Na, ²⁶ Al, ⁴⁰K, ⁸³ Sr, or ¹²⁴I. In embodiments, the positron-emitting radioisotope is ¹¹C. In embodiments, the positron-emitting radioisotope is ¹³N. In embodiments, the positron-emitting radioisotope is ¹⁵O. In embodiments, the positron-emitting radioisotope is ¹⁸F. In embodiments, the positron-emitting radioisotope is ⁶⁴Cu. In embodiments, the positron-emitting radioisotope is ¹⁶⁸ Ga. In embodiments, the positron-emitting radioisotope is ⁷⁸Br. In embodiments, the positron-emitting radioisotope is ⁸²Rb. In embodiments, the positron-emitting radioisotope is ⁸⁶Y. In embodiments, the positron-emitting radioisotope is ⁸⁹Zr. In embodiments, the positron-emitting radioisotope is ⁹⁰Y. In embodiments, the positron-emitting radioisotope is ²²Na. In embodiments, the positron-emitting radioisotope is ²⁶ Al. In embodiments, the positron-emitting radioisotope is ⁴⁰K. In embodiments, the positron-emitting radioisotope is ⁸³Sr. In embodiments, the positron-emitting radioisotope is ¹²⁴I. In embodiments, the radioisotope is an alpha-emitting radioisotope. In embodiments, the alpha-emitting radioisotope is ²¹¹ At, ²²⁷Th, ²²⁵ Ac, ²²³Ra, ²¹³Bi, or ²¹²Bi. In embodiments, the alpha-emitting radioisotope is ²¹¹At. In embodiments, the alpha-emitting radioisotope is ²²⁷Th. In embodiments, the alpha-emitting radioisotope is ²²⁵ Ac. In embodiments, the alpha-emitting radioisotope is ²²³Ra. In embodiments, the alpha-emitting radioisotope is ²¹³ Bi. In embodiments, the alpha-emitting radioisotope is ²¹²Bi.

The term “therapeutic agent” refers to any agent useful in treating and/or preventing a disease. “Therapeutic agent” includes, without limitation, small molecule drugs, proteins, nucleic acids (e.g., DNA, RNA), and the like. “Small-molecule drugs” refers to chemical compounds with low molecular weight that are capable of treating and/or preventing diseases. In embodiments, the proteins described herein are bonded to a therapeutic agent. In embodiments, an antibody or antibody variant is bonded to a therapeutic agent. In embodiments, a nanobody is bonded to a therapeutic agent. In embodiments, the bond is noncovalent or covalent. In embodiments, the bond is covalent. In embodiments, the protein is covalently bonded to a therapeutic agent. In embodiments, the antibody or antibody variant is covalently bonded to a therapeutic agent. In embodiments, a nanobody is covalently bonded to a therapeutic agent. In embodiments when the protein is covalently bonded to a therapeutic agent, the covalent bond is between the therapeutic agent and a naturally-occurring amino acid in the protein. In embodiments when the nanobody is covalently bonded to a therapeutic agent, the covalent bond is between the therapeutic agent and a naturally-occurring amino acid in the nanobody. Methods for covalently bonding therapeutic agents to proteins are well-known in the art.

### Substituents

In embodiments of the proteins, described herein, R¹ is hydrogen, halogen, —CX¹₃, —CHX¹₂, —CH₂X¹, —OCX¹₃, —OCH₂X¹, —OCHX¹₂, —CN, —SOMIR^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —NR₃⁺, substituted or unsubstituted alkyl, substituted or unsubstituted heteroalkyl, substituted or unsubstituted aryl, or substituted or unsubstituted heteroaryl. In embodiments, R¹ is hydrogen, halogen, —CX¹₃, —CHX¹₂, —CH₂X¹, —OCX¹₃, —OCH₂X¹, —OCHX¹₂, —CN, —SOMIR^{1A}, —SOᵥ₁NR^{1A}R^{1B} —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A} —NR^{1A}SO₂R^{1B}, —NR^{1A}C(O)R^{1B}, —NR^{1A}C(O)OR^{1B}, —NR^{1A}OR^{1B}, —NR₃⁺, substituted or unsubstituted alkyl, or substituted or unsubstituted heteroalkyl. In embodiments, R¹ is halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, —NR₃⁺, substituted or unsubstituted alkyl, or substituted or unsubstituted heteroalkyl.

In embodiments, R¹ is an electron-donating group or an electron-withdrawing group.

In embodiments, R¹ is an electron-withdrawing group. In embodiments, the electron-withdrawing group is halogen, —CX¹₃, —CHX¹₂, —CH₂X¹, —CN, —SOₙ₁R^{1A}, —SOᵥ₁NRIAR^{1B}, —N(O)ₘ₁, —C(O)R^{1A}, —C(O)OR^{1A}, —C(O)NR^{1A}R^{1B}, —NR^{1A}OR^{1B}, —NR₃⁺, substituted or unsubstituted alkyl, or substituted or unsubstituted heteroalkyl; wherein X¹, R^{1A}, R^{1B}, n1, v1, and m1 are as defined herein. In embodiments, R^{1A} and R^{1B} are hydrogen.

In embodiments, R¹ is an electron-donating group. In embodiments, the electron-donating group is —Cl, —Br, —I, —CX²₃, —CHX²₂, —OCX¹₃, —OCH₂X¹, —OCHX¹₂, —OCOR^{1A}, —OC(O)R^{1A}, —OC(O)NR^{1A}R^{1B}, —SR^{1A}, —PR^{1A}R^{1B}—NHC(O)NR^{1A}R^{1B}, —NR^{1A}R^{1B}, —OR^{1A}, —NR^{1A}SO₂R^{1B}, —NR^{1A}C(O)R^{1B}, —NR^{1A}C(O)OR^{1B}, substituted or unsubstituted alkyl, substituted or unsubstituted heteroalkyl, substituted or unsubstituted aryl, or substituted or unsubstituted heteroaryl. In embodiments, the substituted or unsubstituted alkyl is substituted or unsubstituted alkene. In embodiments, the electron-donating group is unsubstituted alkene. In embodiments, the substituted or unsubstituted alkyl is substituted or unsubstituted alkyne. In embodiments, R^{1A} and R^{1B} are hydrogen. In embodiments, the electron-donating group is unsubstituted alkyne.

In embodiments of the proteins described herein, R¹ is substituted or unsubstituted heteroalkyl. In embodiments, R¹ is unsubstituted heteroalkyl. In embodiments, R¹ is unsubstituted 2 to 8 membered heteroalkyl. In embodiments, R¹ is unsubstituted 2 to 6 membered heteroalkyl. In embodiments, R¹ is unsubstituted 2 to 4 membered heteroalkyl. In embodiments, R¹ is —O(CH₂)ₘCH₃, and m is an integer from 0 to 6. In embodiments, R¹ is —O(CH₂)ₘCH₃, and m is an integer from 0 to 4. In embodiments, R¹ is —O(CH₂)ₘCH₃, and m is an integer from 0 to 3. In embodiments, R¹ is —O(CH₂)ₘCH₃, and m is an integer from 0 to 2. In embodiments, R¹ is —O(CH₂)ₘCH₃, and m is 0 or 1. In embodiments, R¹ is —OCH₃. In embodiments, R¹ is —OCH₂CH₃, In embodiments, R¹ is —O(CH₂)₂CH₃, In embodiments, R¹ is —O(CH₂)₃CH₃. In embodiments, R¹ is hydrogen.

In embodiments of the proteins described herein, R¹ is halogen. In embodiments, R¹ is fluorine, chlorine, bromine, or iodine. In embodiments, R¹ is fluorine, chlorine, or bromine. In embodiments, R¹ is fluorine or chlorine. In embodiments, R¹ is fluorine or bromine. In embodiments, R¹ is chlorine or bromine. In embodiments, R¹ is fluorine. In embodiments, R¹ is chlorine. In embodiments, R¹ is bromine. In embodiments, R¹ is iodine.

In embodiments, R¹ is —CX¹₃, —CHX¹₂, or —CH₂X¹, wherein X¹ is halogen. In embodiments, R¹ is —CH₂X¹. In embodiments, R¹ is —CHX¹₂. In embodiments, R¹ is —CX¹₃. In embodiments, R¹ is —CF₃. In embodiments, R¹ is —CHF₂. In embodiments, R¹ is —CH₂F. In embodiments, R¹ is —CCl₃. In embodiments, R¹ is —CHCI₂. In embodiments, R¹ is —CH₂Cl. In embodiments, R¹ is —CBr₃. In embodiments, R¹ is —CHBr₂. In embodiments, R¹ is —CH₂Br. In embodiments, R¹ is —CN. In embodiments, R¹ is —N(O)ₘ₁. In embodiments, R¹ is —NO₂. In embodiments, R¹ is —SOₙ₁R^{1A}. In embodiments, R¹ is —SO₂H. In embodiments, R¹ is —SOᵥ₁NR^{1A}R^{1B} In embodiments, R¹ is —SO₂NH₂. In embodiments, R¹ is —NR₃⁺.

In embodiments of the proteins described herein, R¹ is an alkyl group substituted with an electron-withdrawing group. In embodiments, R¹ is a halogen-substituted alkyl group. In embodiments, —(CH₂)_{w}CX¹₃, —(CH₂)_{w}CHX¹₂, or —(CH₂)_{w}CH₂X¹, wherein w is an integer from 1 to 5, and X¹ is halogen. In embodiments, w is 1. In embodiments, w is 2. In embodiments, w is 3. In embodiments, w is 4. In embodiments, w is 5.

In embodiments of the proteins described herein, R^{1A} is hydrogen, substituted or unsubstituted alkyl, or substituted or unsubstituted heteroalkyl. In embodiments, RIA is hydrogen, unsubstituted alkyl, or unsubstituted heteroalkyl. In embodiments, R^{1A} is hydrogen, substituted or unsubstituted C₁₋₄ alkyl, or substituted or unsubstituted 2 to 4 membered heteroalkyl. In embodiments, R^{1A} is hydrogen, unsubstituted C₁₋₄ alkyl, or unsubstituted 2 to 4 membered heteroalkyl. In embodiments, R^{1A} is hydrogen. In embodiments, R^{1A} is unsubstituted C₁₋₄ alkyl. In embodiments, R^{A} is unsubstituted 2 to 4 membered heteroalkyl. In embodiments, R^{1A} is hydrogen and R^{1B} is hydrogen.

In embodiments of the proteins described herein, R^{1B} is hydrogen, substituted or unsubstituted alkyl, or substituted or unsubstituted heteroalkyl. In embodiments, R^{1B} is hydrogen, unsubstituted alkyl, or unsubstituted heteroalkyl. In embodiments, R^{1B} is hydrogen, substituted or unsubstituted C₁₋₄ alkyl, or substituted or unsubstituted 2 to 4 membered heteroalkyl. In embodiments, R^{1B} is hydrogen, unsubstituted C₁₋₄ alkyl, or unsubstituted 2 to 4 membered heteroalkyl. In embodiments, R^{1B} is hydrogen. In embodiments, R^{1B} is unsubstituted C₁₋₄ alkyl. In embodiments, R^{1B} is unsubstituted 2 to 4 membered heteroalkyl. In embodiments, R^{1A} is hydrogen and R^{1B} is hydrogen.

In embodiments of the proteins described herein, X¹ is independently-F, —Cl, —Br, or —I. In embodiments, X¹ is independently —F, —Cl, or —Br. In embodiments, X¹ is independently —F or —Cl. In embodiments, X¹ is —F. In embodiments, X¹ is —Cl. In embodiments, X¹ is —Br. In embodiments, X¹ is —I.

In embodiments of the proteins described herein, n1 is an integer from 0 to 4. In embodiments n1 is an integer from 0 to 3. In embodiments n1 is an integer from 0 to 2. In embodiments n1 is 0. In embodiments n1 is 1. In embodiments n1 is 2. In embodiments n1 is 3. In embodiments n1 is 4.

In embodiments of the proteins described herein, m1 is 1 or 2. In embodiments, m1 is 1. In embodiments, m1 is 2.

In embodiments of the proteins described herein, v1 is 1 or 2. In embodiments, v1 is 1. In embodiments, v1 is 2.

In embodiments of the proteins described herein, x is an integer from 0 to 8. In embodiments, x is an integer from 1 to 8. In embodiments, x is an integer from 1 to 7. In embodiments, x is an integer from 1 to 6. In embodiments, x is an integer from 1 to 5. In embodiments, x is an integer from 1 to 4. In embodiments, x is an integer from 1 to 3. In embodiments, x is an integer of 1 or 2. In embodiments, x is 1. In embodiments, x is 2. In embodiments, x is 3. In embodiments, x is 4. In embodiments, x is 5. In embodiments, x is 6. In embodiments, x is 7. In embodiments, x is 8. In embodiments, x is 0.

In embodiments of the proteins described herein, L¹ is a bond, substituted or unsubstituted alkylene, or substituted or unsubstituted heteroalkylene. In embodiments, L¹ is a bond. In embodiments, L¹ is substituted or unsubstituted alkylene. In embodiments, L¹ is substituted or unsubstituted C₁₋₆ alkylene. In embodiments, L¹ is substituted or unsubstituted C₁-4 alkylene. In embodiments, L¹ is unsubstituted alkylene. In embodiments, L¹ is unsubstituted C₁₋₆ alkylene. In embodiments, L¹ is unsubstituted C₁₋₄ alkylene. In embodiments, L¹ is methylene. In embodiments, L¹ is ethylene. In embodiments, L¹ is propylene. In embodiments, L¹ is substituted or unsubstituted heteroalkylene. In embodiments, L¹ is substituted or unsubstituted 2 to 8 membered heteroalkylene. In embodiments, L¹ is substituted or unsubstituted 2 to 6 membered heteroalkylene. In embodiments, L¹ is —NH—C(O)—(CH₂)_{y}— or —NH—C(O)—O—(CH₂)_{y}—, and y is an integer from 0 to 6. In embodiments, L¹ is —NH—C(O)—(CH₂)_{y}—or —NH—C(O)—O—(CH₂)_{y}—, and y is an integer from 0 to 5. In embodiments, L¹ is —NH—C(O)—(CH₂)_{y}— or —NH—C(O)—O—(CH₂)_{y}—, and y is an integer from 0 to 4. In embodiments, L¹ is —NH—C(O)—(CH₂)_{y}— or —NH—C(O)—O—(CH₂)_{y}—, and y is an integer from 0 to 3. In embodiments, L¹ is —NH—C(O)—(CH₂)_{y}— or —NH—C(O)—O—(CH₂)_{y}—, and y is an integer from 0 to 2. In embodiments, L¹ is —NH—C(O)—(CH₂)_{y}—, and y is an integer from 0 to 3. In embodiments, L¹ is —NH—C(O)—. In embodiments, L¹ is —NH—C(O)—(CH₂)— In embodiments, L¹ is —NH—C(O)—(CH₂)₂—. In embodiments, L¹ is —NH—C(O)—(CH₂)₃—. In embodiments, L¹ is —NH—C(O)—O—(CH₂)_{y}—, and y is an integer from 0 to 3. In embodiments, L¹ is —NH—C(O)—O—. In embodiments, L¹ is —NH—C(O)—O—(CH₂)—. In embodiments, L¹ is —NH—C(O)—O—(CH₂)₂—. In embodiments, L¹ is —NH—C(O)—O—(CH₂)₃—.

In embodiments of the proteins described herein, —(CH₂)ₓ-L¹- is —(CH₂)ₓNHC(O)— or (CH₂)ₓNHC(O)O—, where x is as defined herein. In embodiments, —(CH₂)ₓ-L¹- is (CH₂)ₓNHC(O)—, where x is as defined herein. In embodiments, —(CH₂)ₓ-L¹-is —(CH₂) NHC(O)—. In embodiments, —(CH₂)ₓ-L¹- is —(CH₂)₂NHC(O)—. In embodiments, —(CH₂)-L¹- is —(CH₂)₃NHC(O)—. In embodiments, —(CH₂)ₓ-L¹- is —(CH₂)₄NHC(O)—. In embodiments, (CH₂)ₓ-L¹- is —(CH₂)₃NHC(O)—. In embodiments, —(CH₂)ₓ-L¹- is —(CH₂)₆NHC(O)—. In embodiments, —(CH₂)ₓ-L¹- is —(CH₂)ₓNHC(O)O—, where x is as defined herein. In embodiments, (CH₂)ₓ-L¹- is —(CH₂) NHC(O)O—. In embodiments, —(CH₂)ₓ-L¹- is —(CH₂) 2NHC(O)O—. In embodiments, —(CH₂)ₓ-L¹- is —(CH₂) 3NHC(O)O—. In embodiments, —(CH₂)ₓ-L¹- is
(CH₂)₄NHC(O)O—. In embodiments, —(CH₂)ₓ-L¹- is —(CH₂)₅NHC(O)O—. In embodiments,
(CH₂)ₓ-L¹- is —(CH₂)₆NHC(O)O—.

### Protein-Protein Conjugates

The disclosure provides a method of covalently binding a protein as described herein (including embodiments thereof) to a target protein (as described herein) comprising contacting the protein as described herein (e.g., having an unnatural amino acid and a non-naturally or naturally occurring arginine proximal to the unnatural amino acid) with a lysine, histidine, or tyrosine of a target protein, thereby covalently bonding the protein to the target protein. In embodiments, the protein is an antibody or antibody variant. The resulting covalent bond between the protein and target protein is represented below.

The bond between the unnatural amino acid side chain (—S(O₂)—) of the protein described herein and the lysine in the target protein is represented as follows:

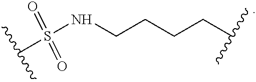 [SMILES: *CCCCNS(*)(=O)=O]

The bond between the unnatural amino acid side chain (—S(O₂)—) of the protein described herein and the histidine in the target protein is represented as follows:

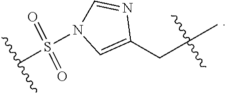 [SMILES: *Cc1cn(S(*)(=O)=O)cn1]

The bond between the unnatural amino acid side chain (—S(O₂)—) of the protein described herein and the tyrosine in the target protein is represented as follows:

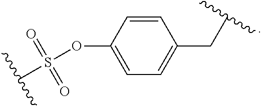 [SMILES: *Cc1ccc(OS(*)(=O)=O)cc1]

In embodiments, the target protein is a cell surface receptor, a cytosolic protein, a transcriptional factor, or an enzyme. In embodiments, the cell surface receptor is in the extracellular domain, the transmembrane domain, or the intracellular domain. In embodiments, the target protein is a programmed death-ligand 1 (PD-L1) receptor, a programmed cell death protein 1 (PD-1) receptor, a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor (EGFR), a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SIP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, or a vasopressin receptor. In embodiments, the target protein is a PD-L1 receptor or a PD-1 receptor. In embodiments, the target protein is a receptor expressed on a cancer cell. In embodiments, wherein the target protein is a G protein-coupled receptor. In embodiments, the target protein is a receptor tyrosine kinase. In embodiments, the target protein is an ErbB receptor. In embodiments, the target protein is an epidermal growth factor receptor

### Cells

In embodiments, the disclosure provides a cell comprising the proteins described herein. In embodiments, the cell further comprises a vector as described herein. In embodiments, the protein described herein, including embodiments thereof, is biosynthesized inside the cell, thereby generating a cell containing the protein. In embodiments, the protein described herein, including embodiments thereof, is contained in the medium outside the cell and penetrates into the cell, thereby generating a cell containing the protein. A cell can be any prokaryotic or eukaryotic cell. For example, any of the proteins described herein can be expressed in bacterial cells such as E. coli, insect cells, yeast or mammalian cells (such as Hela cells, Chinese hamster ovary cells (CHO) or COS cells). In embodiments, a cell can be a premature mammalian cell, i.e., pluripotent stem cell. In embodiments, a cell can be derived from other human tissue. A cell can be any prokaryotic or eukaryotic cell. In embodiments, the cell is prokaryotic. In embodiments, the cell is eukaryotic. In embodiments, the cell is a bacterial cell, a fungal cell, a plant cell, an archael cell, or an animal cell. In embodiments, the animal cell is an insect cell or a mammalian cell. In embodiments, the cell is a bacterial cell. In embodiments, the cell is a fungal cell. In embodiments, the cell is a plant cell. In embodiments, the cell is an archael cell. In embodiments, the cell is an animal cell. In embodiments, the cell is an insect cell. In embodiments, the cell is a mammalian cell. In embodiments, the cell is a human cell. For example, any of the compositions described herein can be expressed in bacterial cells such as E. coli, insect cells, yeast or mammalian cells (such as Hela cells, Chinese hamster ovary cells (CHO) or COS cells). In embodiments, the cell is a premature mammalian cell, i.e., a pluripotent stem cell. In embodiments, the cell is derived from other human tissue. Other suitable cells are known to those skilled in the art.

The proteins provided herein may be delivered to cells using methods well known in the art. Provided herein is a nucleic acid sequence encoding the proteins described herein, including embodiments thereof. Provided herein is a vector comprising a nucleic acid sequence encoding the protein described herein, including embodiments thereof.

### Pharmaceutical Compositions

Any of the proteins described herein may be administered to a subject in a pharmaceutical composition further comprising a pharmaceutically acceptable excipient. The compositions are suitable for formulation and administration in vitro or in vivo. Suitable carriers and excipients and their formulations are known in the art and described, e.g., Remington: The Science and Practice of Pharmacy, 21st Ed, Lippicott Williams & Wilkins (2005).

The term “pharmaceutical composition” encompasses compositions administered to a patient for therapeutic purposes (e.g., treating a disease) and/or diagnostic purposes (e.g., medical imaging). Medical imagining includes, without limitation, radiography, magnetic resonance imaging, nuclear medicine, ultrasound elastography, photoacoustic imaging, tomography (e.g., positron emission tomography), echocardiography, functional near-infrared spectroscopy, magnetic particle imaging, and the like.

“Pharmaceutically acceptable excipient” and “pharmaceutically acceptable carrier” refer to a substance that aids the administration of an active agent to and absorption by a subject and can be included in the compositions of the disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful. Pharmaceutically acceptable excipients can be used in pharmaceutical compositions for therapeutic purposes (e.g., treating a disease) and/or diagnostic purposes (e.g., imaging, such as positron emission tomography).

Solutions of the pharmaceutical compositions can be prepared in water suitably mixed with a lipid or surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms. Solutions can be administered, e.g., parenterally, such as subcutaneously or intravenously (e.g., infusion or bolus).

Pharmaceutical compositions can be delivered via intranasal or inhalable solutions. The intranasal composition can be a spray, aerosol, or inhalant. The inhalable composition can be a spray, aerosol, or inhalant. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known in the art.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or edible carrier, or they may be enclosed in hard or soft shell gelatin capsule, or they may be compressed into tablets, or they may be incorporated directly with the food. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may be between about 1 to about 75% of the weight of the unit. The amount of nucleic acids in such compositions is such that a suitable dosage can be obtained.

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 m1 of isotonic NaCl solution and either added to 1000 m1 of hypodermoclysis fluid or injected at the proposed site of infusion.

Sterile injectable solutions can be prepared by incorporating the recombinant proteins in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. Dimethyl sulfoxide can be used as solvent for rapid penetration, delivering high concentrations of the active agents to a small area.

For vaccination or immunization purposes the proteins described herein may be formulated and introduced as a vaccine through oral, intradermal, intramuscular, intraperitoneal, intravenous, subcutaneous, intranasal, and via scarification (scratching through the top layers of skin, e.g., using a bifurcated needle) or any other standard route of immunization. Vaccine formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient or any other oral composition as listed above. Alternatively, the vaccines may be administered parenterally as injections (intravenous, intramuscular or subcutaneous). The amount of recombinant proteins used in a vaccine can depend upon a variety of factors including the route of administration, species, and use of booster administration. However, a person of ordinary skill in the art would immediately recognize appropriate and/or equivalent doses looking at dosages of approved whopping cough vaccines for guidance.

The term “adjuvant” refers to a compound that when administered in conjunction with the recombinant proteins provided herein including embodiments thereof augments the immune response to the antigen, but when administered alone does not generate an immune response to the antigen. As described above the recombinant proteins provided herein including embodiments thereof may be used as an adjuvant. Therefore, the term “adjuvant” refers to a compound that when administered in conjunction with a vaccine augments the immune response to the antigen, but when administered alone does not generate an immune response to the antigen. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the recombinant proteins provided herein to elicit an immune response. Adjuvants augment the intrinsic response to an immunogen without causing conformational changes in the immunogen that affect the qualitative form of the response. Adjuvants can be administered as a component of a therapeutic composition with an active agent or can be administered separately, before, concurrently with, or after administration of the therapeutic agent.

An adjuvant can be administered with an immunogen as a single composition, or can be administered before, concurrent with or after administration of the immunogen. Immunogen and adjuvant can be packaged and supplied in the same vial or can be packaged in separate vials and mixed before use. Immunogen and adjuvant are typically packaged with a label indicating the intended therapeutic application. If immunogen and adjuvant are packaged separately, the packaging typically includes instructions for mixing before use. The choice of an adjuvant and/or carrier depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Alum, MPL and QS-21 are preferred. Optionally, two or more different adjuvants can be used simultaneously. Preferred combinations include alum with MPL, alum with QS-21, MPL with QS-21, MPL or RC-529 with GM-CSF, and alum, QS-21 and MPL together. Also, Incomplete Freund's adjuvant can be used (Chang et al., Advanced Drug Delivery Reviews 32, 173-186 (1998)), optionally in combination with any of alum, QS-21, and MPL and all combinations thereof.

### Dose and Dosing Regimens

The dosage and frequency (single or multiple doses) of the proteins described herein administered to a subject can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and proteins described herein. Adjustment and manipulation of established dosages (e.g., frequency and duration) are within the ability of the skilled artisan.

For any composition, the effective amount of a protein described herein can be initially determined from cell culture assays. Target concentrations will be those concentrations of protein that are capable of achieving the methods described herein, as measured using the methods described herein or known in the art. As is known in the art, effective amounts of proteins for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages of the proteins described herein may be varied depending upon the requirements of the patient, and whether the purpose is therapeutic or medical imaging. The dose administered to a patient should be sufficient to affect a beneficial therapeutic response in the patient over time. The size of the dose also will be determined by the existence, nature, and extent of any adverse side-effects. Determination of the proper dosage for a particular situation is within the skill of the art. Dosage amounts and intervals can be adjusted individually to provide levels of the protein effective for the particular clinical indication being treated. This will provide a therapeutic regimen that is commensurate with the severity of the individual's disease state.

Utilizing the teachings provided herein, an effective prophylactic, diagnostic, or therapeutic treatment regimen can be planned that does not cause substantial toxicity and yet is effective to treat the clinical disease or symptoms demonstrated by the particular patient. This planning should involve the careful choice of proteins by considering factors such as compound potency, relative bioavailability, patient body weight, presence and severity of adverse side effects.

In embodiments, the proteins are administered to a patient at an amount of about 0.001 mg/kg to about 500 mg/kg. In embodiments, the proteins (e.g., recombinant proteins, antibodies, antibody variants, single-domain antibodies) are administered to a patient in an amount of about 0.01 mg/kg, 0.1 mg/kg, 0.5 mg/kg, 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg, or 300 mg/kg. It is understood that where the amount is referred to as “mg/kg,” the amount is milligram per kilogram body weight of the subject being administered with the proteins. In embodiments, the proteins are administered to a patient in an amount from about 0.01 mg to about 500 mg per day.

### Embodiments 1-119

Embodiment 1. A protein comprising: (i) an unnatural amino acid, and (ii) a non-naturally occurring arginine; wherein: (a) the non-naturally occurring arginine is proximal to the —S(O₂) F group in the unnatural amino acid side chain; and (b) the unnatural amino comprises a side chain of Formula (I):

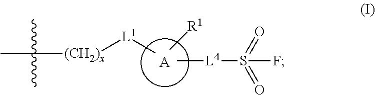

wherein: ring A is phenyl, a 5-membered cycloalkyl, a 5-membered heterocycloalkyl, or a 5-membered heteroaryl; L⁴ is a bond or —O—; x is an integer from 0 to 8; L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹₂, —CN, —SOMIR^{1A}, —SOᵥ₁NR^{1A}R^{1B}, —NHC(O)NR^{1A}R^{1B}, —N(O)ₘ₁, —NR^{1A}R^{1B}, —C(O)R¹⁴, —C(O)—OR14, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently-F, —Cl, —Br, or —I; RIA is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

Embodiment 2. A protein comprising: (i) an unnatural amino acid, and (ii) a naturally occurring arginine; wherein: (a) the arginine is proximal to the —S(O₂) F group in the unnatural amino acid side chain; and (b) the unnatural amino comprises a side chain of Formula (I):

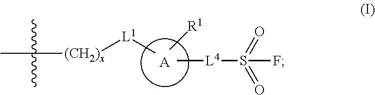

wherein: ring A is phenyl, a 5-membered cycloalkyl, a 5-membered heterocycloalkyl, or a 5-membered heteroaryl; L⁴ is a bond or —O—; x is an integer from 0 to 8; L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O) MI, —NR^{1A}R^{1B}, —C(O)R^{1A}, —C(O)—OR14, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR 1B substituted or unsubstituted alkyl, or substituted or unsubstituted heteroalkyl; X¹ is independently —F, —CL, —BR, OR —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2; provided that when A is phenyl, then the protein is not epidermal growth factor receptor, protein tyrosine phosphatase 1B, P-selectin glycoprotein ligand 1, complement component 5a receptor, chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377.

Embodiment 3. The protein of embodiment 1 or 2, wherein the unnatural amino comprises a side chain of Formula (II):

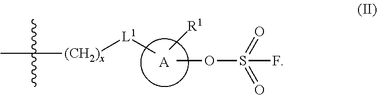

Embodiment 4. The protein of embodiment 1 or 2, wherein the unnatural amino comprises a side chain of Formula (V):

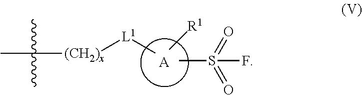

Embodiment 5. The protein of any one of embodiments 1 to 4, wherein R¹ is halogen, —CX¹₃, —CHX¹₂, —CH₂X¹, —OCX¹₃, —OCH₂X¹, —OCHX¹₂, —CN, —SOM1R¹⁴, —SOᵥ₁NR^{1A}R^{1B}, —NHC(O)NR^{1A}R^{1B}, —N(O)^{m1}, —NR^{1A}R^{1B}, —C(O)R^{1A}, —C(O)—OR14, —C(O)NR^{1A}R^{1B}, —OR^{1A}, —NR^{1A}SO₂R^{1B}, —NR 1AC(O)R^{1B}, —NR^{1A}C(O)OR^{1B}, —NR^{1A}OR^{1B}, unsubstituted C₁₋₈ alkyl, or unsubstituted 2 to 8 membered heteroalkyl; RIA is hydrogen, unsubstituted C₁₋₄ alkyl, or unsubstituted 2 to 4 membered heteroalkyl; and R^{1B} is hydrogen, unsubstituted C₁₋₄ alkyl, or unsubstituted 2 to 4 membered heteroalkyl.

Embodiment 6. The protein of embodiment 5, wherein R¹ is halogen.

Embodiment 7. The protein of any one of embodiments 1 to 6, wherein R¹ is ortho to —S(O₂)F.

Embodiment 8. The protein of any one of embodiments 1 to 6, wherein R¹ is meta to —S(O₂)F.

Embodiment 9. The protein of any one of embodiments 1 to 4, wherein the unnatural amino comprises a side chain of Formula (III):

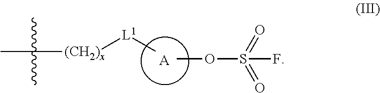

Embodiment 10. The protein of any one of embodiments 1 to 4, wherein the unnatural amino comprises a side chain of Formula (VI):

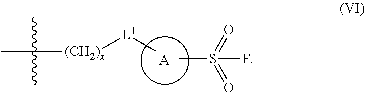

Embodiment 11. The protein of any one of embodiments 1 to 10, wherein ring A is phenyl.

Embodiment 12. The protein of any one of embodiments 1 to 10, wherein ring A is a 5-membered cycloalkyl having one or two double bonds or a 5-membered heterocycloalkyl having one double bond.

Embodiment 13. The protein of any one of embodiments 1 to 10, wherein ring A is a 5-membered cycloalkyl.

Embodiment 14. The protein of any one of embodiments 1 to 10, wherein ring A is a 5-membered heterocycloalkylene.

Embodiment 15. The protein of any one of embodiments 1 to 10, wherein ring A is a 5-membered heteroaryl.

Embodiment 16. The protein of embodiment 15, wherein ring A is a 5-membered heteroaryl containing 1 to 3 heteroatoms selected from the group consisting of oxygen, nitrogen, and sulfur.

Embodiment 17. The protein of embodiment 15, wherein ring A is a 5-membered heteroaryl containing 1 or 2 heteroatoms selected from the group consisting of oxygen, nitrogen, and sulfur.

Embodiment 18. The protein of embodiment 15, wherein ring A is a 5-membered heteroaryl containing 1 heteroatom selected from the group consisting of oxygen, nitrogen, and sulfur.

Embodiment 19. The protein of any one of embodiment 1 to 10, wherein ring A is pyrrole, pyrazole, imidazole, triazole, furan, thiophene, phosphole, oxazole, isoxazole, thiazole, or isothiazole.

Embodiment 20. The protein of any one of embodiments 1 to 19, wherein L¹ is a bond.

Embodiment 21. The protein of any one of embodiments 1 to 19, wherein L¹ is substituted or unsubstituted alkylene.

Embodiment 22. The protein of embodiment 21, wherein L¹ is substituted or unsubstituted C₁₋₄ alkylene.

Embodiment 23. The protein of any one of embodiment 1 to 19, wherein L¹ is substituted or unsubstituted heteroalkylene.

Embodiment 24. The protein of embodiment 23, wherein L¹ is substituted or unsubstituted 2 to 6 membered heteroalkylene.

Embodiment 25. The protein of embodiment 24, wherein L¹ is —NH—C(O)—(CH₂)_{y}—, and y is an integer from 0 to 2.

Embodiment 26. The protein of embodiment 24, wherein L¹ is —NH—C(O)—O—(CH₂)_{y}—, and y is an integer from 0 to 2.

Embodiment 27. The protein of embodiment 24, wherein L¹ is —NH—C(O)—NH—(CH₂)_{y}—, and y is an integer from 0 to 2.

Embodiment 28. The protein of embodiment 24, wherein L¹ is —NH—C(O)—S—(CH₂)_{y}—, and y is an integer from 0 to 2.

Embodiment 29. The protein of any one of embodiments 25 to 28, wherein y is 0.

Embodiment 30. The protein of any one of embodiments 1 to 29, wherein x is an integer from 0 to 6.

Embodiment 31. The protein of embodiment 30, wherein x is an integer from 2 to 6. Embodiment 32. The protein of embodiment 31, wherein x is 4.

Embodiment 33. The protein of any one of embodiments 1 to 19, wherein —(CH₂)ₓ- L¹- is —(CH₂)₄NH—C(O)—.

Embodiment 34. The protein of any one of embodiments 1 to 19, wherein —(CH₂)ₓ- L¹- is —(CH₂)₄NH—C(O)—O—.

Embodiment 35. The protein of any one of embodiments 1 to 19, wherein —(CH₂)ₓ-L¹- is —(CH₂)₄NH—C(O)—NH—.

Embodiment 36. The protein of any one of embodiments 1 to 19, wherein —(CH₂)ₓ-L¹- is —(CH₂)₄NH—C(O)—S—.

Embodiment 37. The protein of embodiment 1 or 2, wherein the unnatural amino comprises a side chain of Formula (FSY):

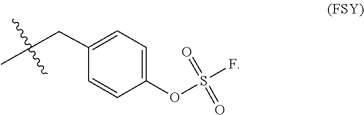 [SMILES: *Cc1ccc(OS(=O)(=O)F)cc1]

Embodiment 38. The protein of embodiment 1 or 2, wherein the unnatural amino comprises a side chain of Formula (mFSY):

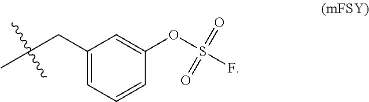 [SMILES: *Cc1cccc(OS(=O)(=O)F)c1]

Embodiment 39. The protein of embodiment 1 or 2, wherein the unnatural amino comprises a side chain of Formula (FFY):

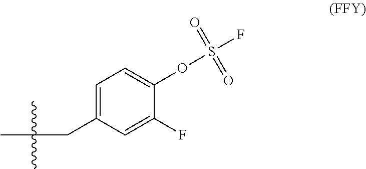 [SMILES: *Cc1ccc(OS(=O)(=O)F)c(F)c1]

Embodiment 40. The protein of embodiment 1 or 2, wherein the unnatural amino comprises a side chain of Formula (FSK):

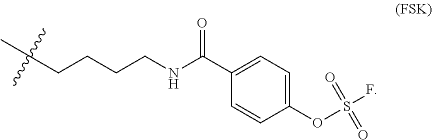 [SMILES: *CCCCNC(=O)c1ccc(OS(=O)(=O)F)cc1]

Embodiment 41. The protein of embodiment 1 or 2, wherein the unnatural amino comprises a side chain of Formula (mFSK):

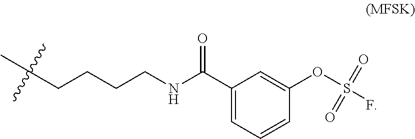 [SMILES: *CCCCNC(=O)c1cccc(OS(=O)(=O)F)c1]

Embodiment 42. The protein of embodiment 1 or 2, wherein the unnatural amino comprises a side chain of Formula (IV):

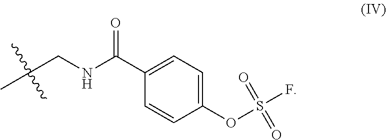 [SMILES: *CNC(=O)c1ccc(OS(=O)(=O)F)cc1]

Embodiment 43. The protein of embodiment 1 or 2, wherein the unnatural amino comprises a side chain of Formula (NHSF):

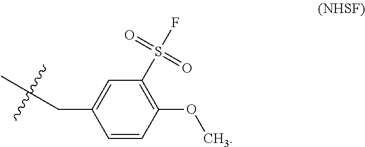 [SMILES: *Cc1ccc(OC)c(S(=O)(=O)F)c1]

Embodiment 44. The protein of embodiment 1 or 2, wherein the unnatural amino comprises a side chain of Formula (VII):

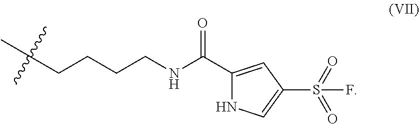 [SMILES: *CCCCNC(=O)c1cc(S(=O)(=O)F)c[nH]1]

Embodiment 45. The protein of embodiment 1 or 2, wherein the unnatural amino comprises a side chain of Formula (VIII):

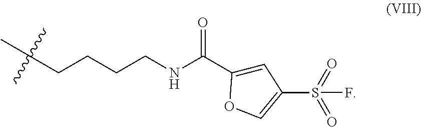 [SMILES: *CCCCNC(=O)c1cc(S(=O)(=O)F)co1]

Embodiment 46. The protein of embodiment 1 or 2, wherein the unnatural amino comprises a side chain of Formula (IX):

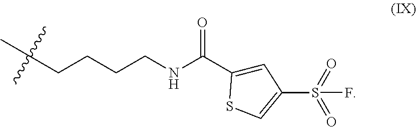 [SMILES: *CCCCNC(=O)c1cc(S(=O)(=O)F)cs1]

Embodiment 47. The protein of embodiment 1 or 2, wherein the unnatural amino comprises a side chain of Formula (X):

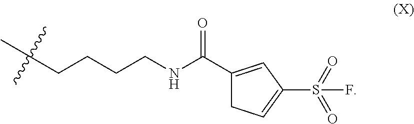 [SMILES: *CCCCNC(=O)C1=CC(S(=O)(=O)F)=CC1]

Embodiment 48. The protein of any one of embodiments 1 to 47, wherein the protein is an antibody.

Embodiment 49. The protein of any one of embodiments 1 to 47, wherein the protein is an antibody variant.

Embodiment 50. The protein of embodiment 49, wherein the antibody variant is a single-chain variable fragment, a single-domain antibody, an affibody, or an antigen-binding fragment.

Embodiment 51. The protein of embodiment 50, wherein the antibody variant is a single-chain variable fragment.

Embodiment 52. The protein of embodiment 50, wherein the antibody variant is a single-domain antibody.

Embodiment 53. The protein of embodiment 50, wherein the antibody variant is an affibody.

Embodiment 54. The protein of embodiment 50, wherein the antibody variant is an antigen-binding fragment.

Embodiment 55. The protein of embodiment any one of embodiments 48 to 54, wherein the unnatural amino acid is within a CDR region of the antibody or antibody variant, and the non-naturally occurring arginine is within a CDR region of the antibody or antibody variant.

Embodiment 56. The protein of embodiment any one of embodiments 48 to 54, wherein the unnatural amino acid is within a CDR region of the antibody or antibody variant, and the non-naturally occurring arginine is within a framework region of the antibody or antibody variant.

Embodiment 57. The protein of embodiment 52, wherein the single-domain antibody comprises an amino acid sequence having at least 90% sequence identity to SEQ ID NO:1.

Embodiment 58. The protein of embodiment 57, wherein the single-domain antibody comprises an amino acid sequence having at least 95% sequence identity to SEQ ID NO:1.

Embodiment 59. The protein of embodiment 58, wherein the single-domain antibody comprises the amino acid sequence of SEQ ID NO:1.

Embodiment 60. The protein of any one of embodiments 57 to 59, wherein the amino acid at the position corresponding to position 44 in SEQ ID NO: 1 is arginine and the position corresponding to position 109 in SEQ ID NO: 1 is FSY.

Embodiment 61. The protein of embodiment 52, wherein the single-domain antibody comprises an amino acid sequence having at least 90% sequence identity to SEQ ID NO:2, provided that the position corresponding to position 44 in SEQ ID NO:2 is arginine and the position corresponding to position 109 in SEQ ID NO:2 is FSY

Embodiment 62. The protein of embodiment 61, wherein the single-domain antibody comprises an amino acid sequence having at least 95% sequence identity to SEQ ID NO:2.

Embodiment 63. The protein of embodiment 62, wherein the single-domain antibody comprises SEQ ID NO:2.

Embodiment 64. The protein of embodiment 52, wherein the single-domain antibody comprises an amino acid sequence having at least 90% sequence identity to SEQ ID NO:8.

Embodiment 65. The protein of embodiment 64, wherein the single-domain antibody comprises an amino acid sequence having at least 95% sequence identity to SEQ ID NO:8.

Embodiment 66. The protein of embodiment 65, wherein the single-domain antibody comprises the amino acid sequence of SEQ ID NO:8.

Embodiment 67. The protein of any one of embodiments 64 to 66, wherein the position corresponding to position 54 in SEQ ID NO:8 is the unnatural amino acid comprising the unnatural amino acid side chain and the position corresponding to position 56 in SEQ ID NO:8 is arginine.

Embodiment 68. The protein of embodiment 52, wherein the single-domain antibody comprises an amino acid sequence having at least 90% sequence identity to SEQ ID NO:9, provided that the position corresponding to position 54 in SEQ ID NO:9 is FSY and the position corresponding to position 56 in SEQ ID NO:9 is arginine.

Embodiment 69. The protein of embodiment 68, wherein the single-domain antibody comprises an amino acid sequence having at least 95% sequence identity to SEQ ID NO:9.

Embodiment 70. The protein of embodiment 69, wherein the single-domain antibody comprises SEQ ID NO:9.

Embodiment 71. The protein of embodiment 53, wherein the affibody comprises an amino acid sequence having at least 90% sequence identity to SEQ ID NO:14.

Embodiment 72. The protein of embodiment 71, wherein the affibody comprises an amino acid sequence having at least 95% sequence identity to SEQ ID NO:14.

Embodiment 73. The protein of embodiment 72, wherein the affibody comprises the amino acid sequence of SEQ ID NO: 14.

Embodiment 74. The protein of any one of embodiments 71 to 73, wherein the position corresponding to position 36 in SEQ ID NO: 14 is the unnatural amino acid comprising the unnatural amino acid side chain and the position corresponding to position 32 in SEQ ID NO:14 is arginine

Embodiment 75. The protein of embodiment 53, wherein the affibody comprises an amino acid sequence having at least 90% sequence identity to SEQ ID NO:15, provided that the position corresponding to position 36 in SEQ ID NO:15 is FSY and the position corresponding to position 32 in SEQ ID NO: 15 is arginine.

Embodiment 76. The protein of embodiment 75, wherein the affibody comprises an amino acid sequence having at least 95% sequence identity to SEQ ID NO:15.

Embodiment 77. The protein of embodiment 76, wherein the affibody comprises SEQ ID NO: 15.

Embodiment 78. The protein of any one of embodiments 1 to 47, wherein the protein is a receptor.

Embodiment 79. The protein of any one of embodiments 1 to 47, wherein the protein is a cell surface receptor.

Embodiment 80. The protein of any one of embodiments 79, wherein the cell surface receptor is in the extracellular domain, the transmembrane domain, or the intracellular domain.

Embodiment 81. The protein of any one of embodiments 1 to 47, wherein the protein is a cytosolic protein.

Embodiment 82. The protein of any one of embodiments 1 to 47, wherein the protein is a transcriptional factor or an enzyme.

Embodiment 83. The protein of any one of embodiments 1 to 82, further comprising a detectable agent.

Embodiment 84. The protein of embodiment 83, wherein the detectable agent is a radioisotope.

Embodiment 85. The protein of any one of embodiments 1 to 84, further comprising a therapeutic agent.

Embodiment 86. A nucleic acid encoding the protein of any one of embodiments 1 to 85.

Embodiment 87. A vector comprising a nucleic acid of embodiment 86.

Embodiment 88. A cell comprising: (i) the protein of any one of embodiments 1 to 85; (ii) the nucleic acid of embodiment 86, or (iii) the vector of embodiment 87.

Embodiment 89. The cell of embodiment 88, wherein the cell is a bacterial cell or a mammalian cell.

Embodiment 90. A pharmaceutical composition comprising: (i) the protein of any one of embodiments 1 to 85, the nucleic acid of embodiment 86, or the vector of embodiment 87; and (ii) a pharmaceutically acceptable excipient.

Embodiment 91. A method of covalently binding a protein to a target protein, the method comprising contacting the protein of any one of embodiments 1 to 85 with a lysine, histidine, or tyrosine of a target protein, thereby covalently binding the protein to the target protein.

Embodiment 92. A method of covalently binding an antibody or an antibody variant to a target protein, the method comprising contacting the antibody or the antibody variant with a lysine, histidine, or tyrosine of a target protein; wherein the antibody or the antibody variant is the protein of any one of embodiments 48 to 77, thereby covalently binding the protein to the target protein.

Embodiment 93. The method of embodiment 91 or 92, wherein the target protein is a receptor.

Embodiment 94. The method of embodiment 93, wherein the receptor is a cell surface receptor.

Embodiment 95. The method of embodiment 94, wherein the cell surface receptor is in the extracellular domain, the transmembrane domain, or the intracellular domain.

Embodiment 96. The method of embodiment 91 or 92, wherein the target protein is a cytosolic protein.

Embodiment 97. The method of embodiment 91 or 92, wherein the target protein is a transcriptional factor.

Embodiment 98. The method of embodiment 91 or 92, wherein the target protein is an enzyme.

Embodiment 99. The method of embodiment 91 or 92, wherein the target protein is a programmed death-ligand 1 receptor, a programmed cell death protein 1 receptor, a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor, a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SIP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, or a vasopressin receptor.

Embodiment 100. The method of embodiment 91 or 92, wherein the target protein is a PD-LI receptor or a PD-1 receptor.

Embodiment 101. The method of embodiment 91 or 92, wherein the target protein is a receptor expressed on a cancer cell.

Embodiment 102. The method of embodiment 91 or 92, wherein the target protein is a G protein-coupled receptor.

Embodiment 103. The method of embodiment 91 or 92, wherein the target protein is a receptor tyrosine kinase.

Embodiment 104. The method of embodiment 91 or 92, wherein the target protein is an ErbB receptor.

Embodiment 105. The method of embodiment 91 or 92, wherein the target protein is an epidermal growth factor receptor

Embodiment 106. A method of covalently binding a single-domain antibody to epidermal growth factor receptor, the method comprising contacting the single domain antibody with the epidermal growth factor receptor; wherein the single domain antibody is the protein of any one of embodiments 57 to 63, thereby covalently binding the single-domain antibody to the epidermal growth factor receptor.

Embodiment 107. The method of embodiment 106, wherein the —S(O₂) F group of the unnatural amino acid side chain in the protein covalently bonds to a lysine, histidine, or tyrosine in the epidermal growth factor receptor; and wherein the non-naturally occurring arginine or the naturally occurring arginine facilitates the —F deparature from the —S(O₂) F group during the reaction forming the covalent bond.

Embodiment 108. A method of covalently binding a single domain antibody to c-2, the method comprising contacting the single domain antibody with the human epidermal growth factor receptor-2; wherein the single domain antibody is the protein of any one of embodiments 64 to 70, thereby covalently binding the single-domain antibody to the human epidermal growth factor receptor-2.

Embodiment 109. The method of embodiment 108, wherein the —S(O₂) F group of the unnatural amino acid side chain in the protein covalently bonds to a lysine, histidine, or tyrosine in the human epidermal growth factor receptor-2; and wherein the non-naturally occurring arginine or the naturally occurring arginine facilitates the —F deparature from the —S(O₂) F group during the reaction forming the covalent bond.

Embodiment 110. A method of covalently binding an affibody to protein Z, the method comprising contacting the affibody with the protein Z; wherein the affibody is the protein of any one of embodiments 71 to 77, thereby covalently binding the affibody to the protein Z.

Embodiment 111. The method of embodiment 110, wherein the —S(O₂) F group of the unnatural amino acid side chain in the protein covalently bonds to a lysine, histidine, or tyrosine in the protein Z; and wherein the non-naturally occurring arginine or the naturally occurring arginine facilitates the —F deparature from the —S(O₂) F group during the reaction forming the covalent bond.

Embodiment 112. A biomolecule conjugate comprising the protein of any one of embodiments 57 to 63 covalently bonded to epidermal growth factor receptor.

Embodiment 113. A biomolecule conjugate comprising the protein of any one of embodiments 64 to 70 covalently bonded to human epidermal growth factor receptor-2.

Embodiment 114. A biomolecule conjugate comprising the protein of any one of embodiments 71 to 77 covalently bonded to protein Z.

Embodiment 115. A method of enhancing the bioreactivity or binding efficacy of a protein, the method comprising: (i) mutating a first amino acid to an unnatural amino acid, and (ii) mutating a second amino acid proximal to the first amino acid to arginine; wherein the unnatural amino comprises a side chain of Formula (I)

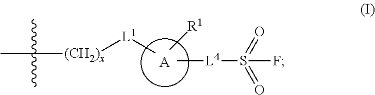

wherein: ring A is phenyl, a 5-membered cycloalkyl, a 5-membered heterocycloalkyl, or a 5-membered heteroaryl; L⁴ is a bond or —O—; x is an integer from 0 to 8; L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹₂, —CN, —SOMIR^{1A}, —N(O)ₘ₁, —SOᵥ₁NR^{1A}R^{1B}, —NHC(O)NR^{1A}R^{1B}, —NR^{1A}R^{1B}, —C(O)R¹⁴, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently-F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

Embodiment 116. The method of embodiment 115, wherein the first amino acid is Arg, Ala, Ile, Leu, Met, Val, Phe, Trp, Tyr, Asn, Cys, Gln, Ser, Thr, Asp, Glu, His, Lys, Gly, or Pro; and wherein the second amino acid is Ala, Ile, Leu, Met, Val, Phe, Trp, Tyr, Asn, Cys, Gln, Ser, Thr, Asp, Glu, His, Lys, Gly, or Pro.

Embodiment 117. A method of enhancing the bioreactivity or binding efficacy of a protein comprising an unnatural amino acid, the method comprising mutating an amino acid proximal to the unnatural amino acid to arginine; wherein the unnatural amino comprises a side chain of Formula (I)

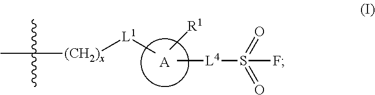

wherein: ring A is phenyl, a 5-membered cycloalkyl, a 5-membered heterocycloalkyl, or a 5-membered heteroaryl; L⁴ is a bond or —O—; x is an integer from 0 to 8; L¹ is a bond, substituted or unsubstituted alkylene, or substituted or unsubstituted heteroalkylene; R¹ is hydrogen, halogen, —CX¹₃, —CHX¹₂, —CH₂X¹, —OCX¹₃, —OCH₂X¹, —OCHX¹₂, —CN, —SOMIR^{1A}, —N(O)ₘ₁, —SOᵥ₁NR^{1A}R^{1B}, —NHC(O)NR^{1A}R^{1B}, —NR^{1A}R^{1B}, —C(O)R^{1A}, —C(O)—OR^{1A}, —C(O)NR^{1A}R^{1B}, —OR14, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl; X¹ is independently —F, —Cl, —Br, or —I; R^{1A} is hydrogen, substituted or unsubstituted alkyl, or substituted or unsubstituted heteroalkyl; R^{1B} is hydrogen, substituted or unsubstituted alkyl, or substituted or unsubstituted heteroalkyl; n1 is an integer from 0 to 4; m1 is 1 or 2; and v1 is 1 or 2.

Embodiment 118. The method of embodiment 117, wherein the amino acid is Ala, Ile, Leu, Met, Val, Phe, Trp, Tyr, Asn, Cys, Gln, Ser, Thr, Asp, Glu, His, Lys, Gly, or Pro

Embodiment 119. The method of any one of embodiments 115 to 118, further comprising contacting the protein with a target protein.

### EXAMPLES

The following examples are intended to further illustrate certain embodiments of the disclosure. The examples are put forth so as to provide one of ordinary skill in the art and are not intended to limit its scope.

The inventors discovered that introducing an arginine (Arg) mutation near a latent bioreactive unnatural amino acid (e.g., such as FSY) in the same protein markedly improved the proximity-enabled sulfur fluoride exchange reaction rate and yield with its target protein. This strategy can be generally applied to different proteins.

The results in the Examples below demonstrate that Arg accelerated the proximity-enabled SuFEx reaction between FSY, mFSY, and FFY and His, Lys, or Tyr incorporated in two binding proteins. The acceleration could be achieved in different proteins.

### Example 1

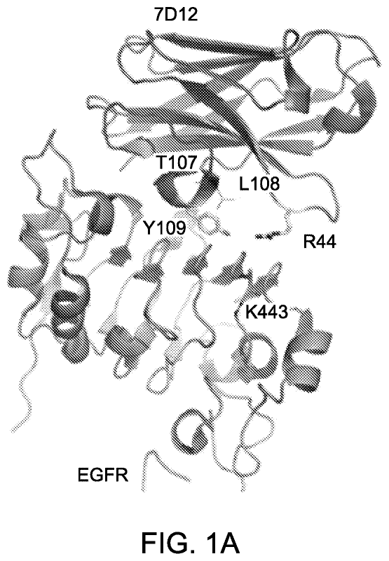

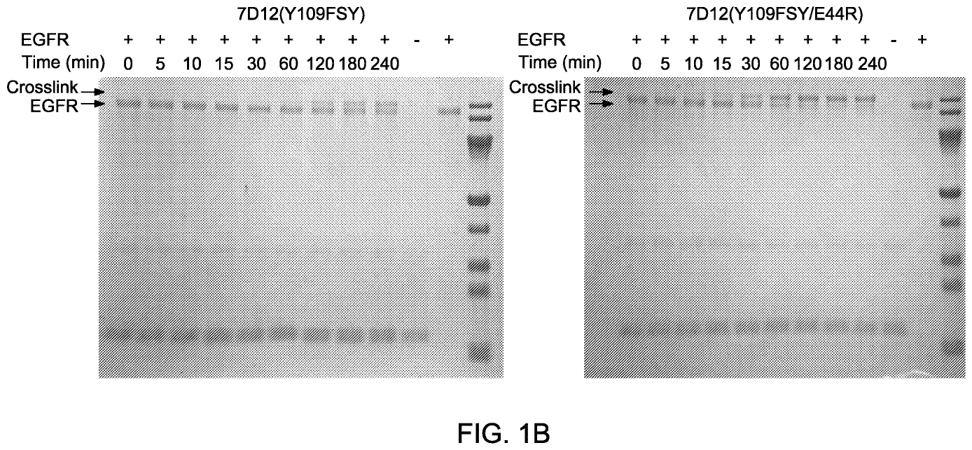

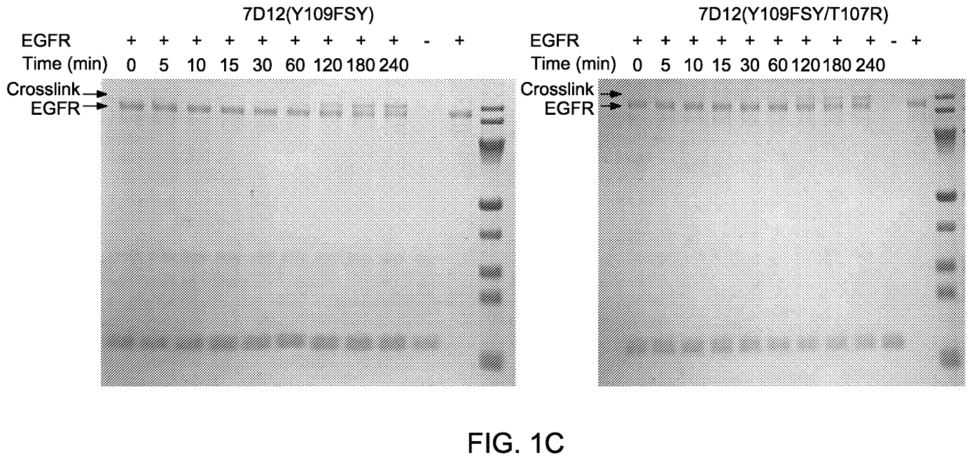

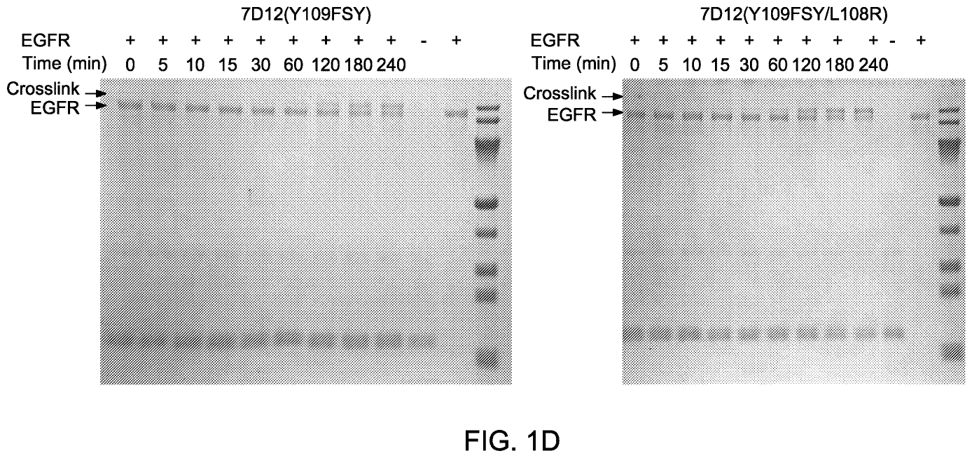

The inventor used nanobody 7D12, which binds to EGFR. On the basis of the crystal structure of 7D12 in complex with EGFR, the inventors mutated Glu44 of 7D12 into Arg, so that the Arg side chain would be close to the side chain of the latent bioreactive Uaa FSY incorporated at site Tyr109 (FIG. 1A). Nanobody 7D12 (Y109FSY/E44R) and control nanobody 7D12 (Y109FSY) were expressed and purified and the from E. coli, and 5 μM of the nanobody protein were individually incubated with 0.4 μM EGFR extracellular domain (ECD) in PBS buffer for crosslinking. At different time points, an aliquot of the reaction mixture was taken and the reaction was stopped by adding SDS-loading buffer. The reaction mixtures were then analyzed on SDS-PAGE to evaluate the crosslinking. As shown in FIG. 1B, for the control nanobody 7D12 (Y109FSY), the crosslinking band could be detected starting 60 min of incubation time. In contrast, the Arg mutant nanobody 7D12 (Y109FSY/E44R) showed crosslinking as soon as 10 min, exhibiting a much fast reaction rate than nanobody 7D12 (Y109FSY). In addition, 7D12 (Y109FSY) crosslinked about 50% of EGFR ECD in 4 h, while 7D12 (Y109FSY/E44R) crosslinked almost all EGFR ECD in 2 h. Therefore, the E44R mutation also increased the crosslinking reaction yield.

The inventors also mutated residues T107 and L¹⁰⁸ to Arg separately, as they are close to Y109 site on ID amino acid sequence. However, introducing Arg at these two sites did not significantly increase the crosslinking rate (FIGS. 1C-1D). These results indicate that the introduced Arg needs to be in an appropriate orientation with FSY in the 3D structure to accelerate the reaction.

### Example 2

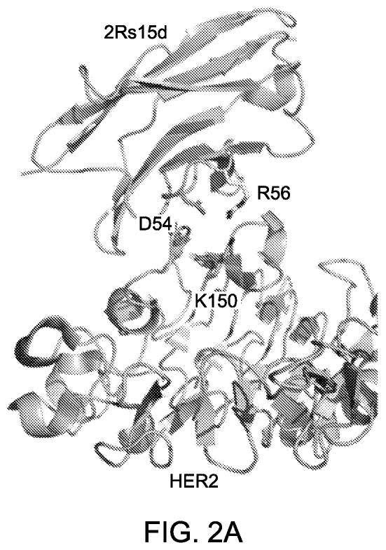

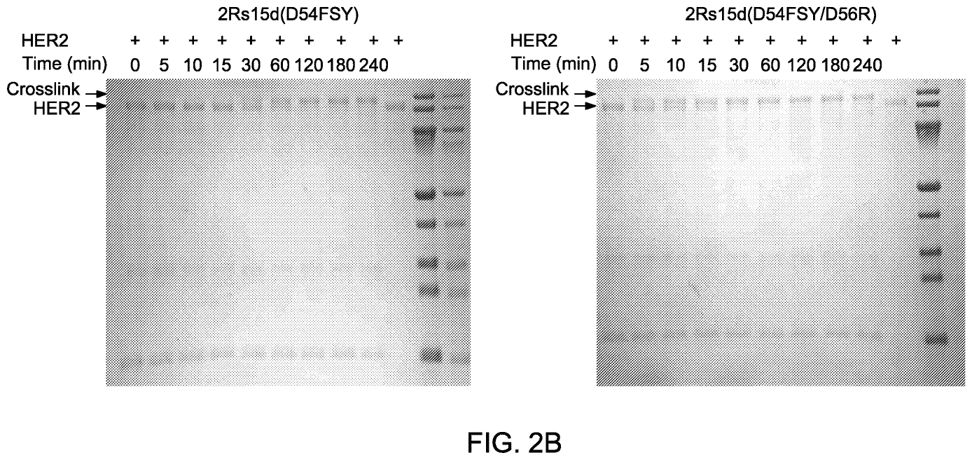

The inventor used nanobody 2Rs15d, which binds to HER2. On the basis of the crystal structure of 2Rs15d in complex with HER2, FSY was incorporated at D54 in the nanobody to target Lys150 of HER2, and Asp56 in 2Rs15d was mutated into Arg (FIG. 2A). Similar procedures were used to express and purify the nanobody proteins 2Rs15d (D54FSY) and 2Rs15d (D54FSY/D56R) from E. coli. These nanobody proteins (5 μM) were individually incubated with 0.4 μM HER2 ECD in PBS buffer for crosslinking, followed with SDS-PAGE at different incubation time points. As shown in FIG. 2B, for 2Rs15d (D54FSY), crosslinking could be detected at about 15 min; in contrast, 2Rs15d (D54FSY/D56R) showed crosslinking as early as 5 min, exhibiting much enhanced crosslinking rate.

Mutation G55R in nanobody 2Rs15d did not increase the crosslinking rate, and mutations S52R and G53R in nanobody 2Rs15d even reduced the crosslinking rate possibly due to steric or ionic interference (data not shown). These results corroborate the importance of appropriate orientation of the introduced Arg side chain toward the FSY side chain in the 3D structure to accelerate the reaction.

### Example 3

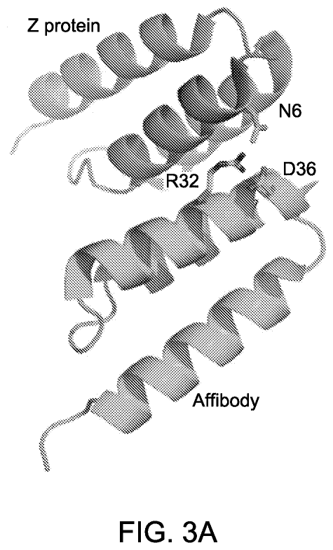

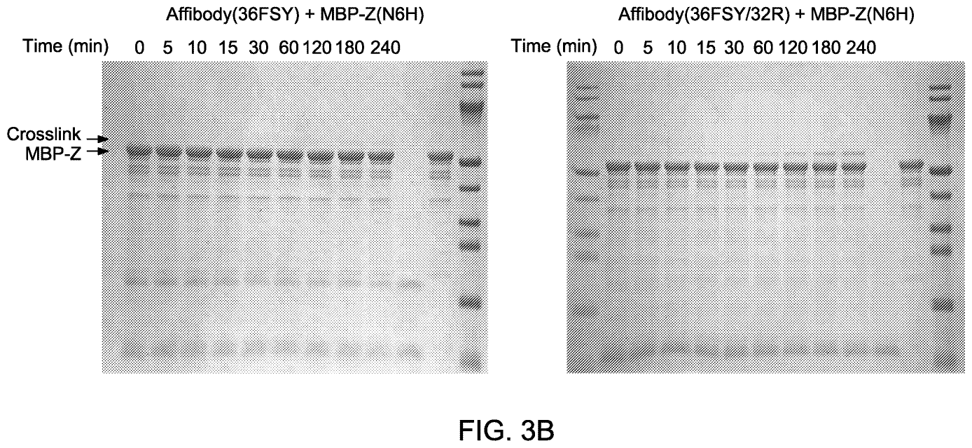

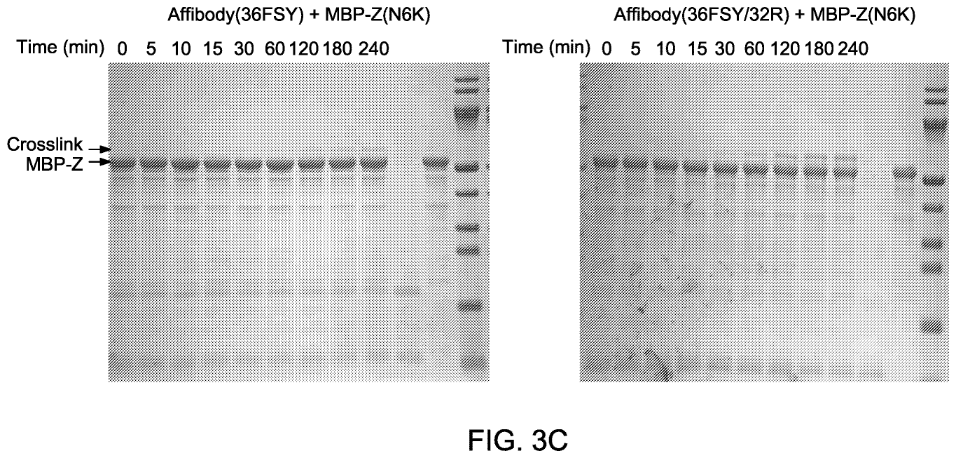

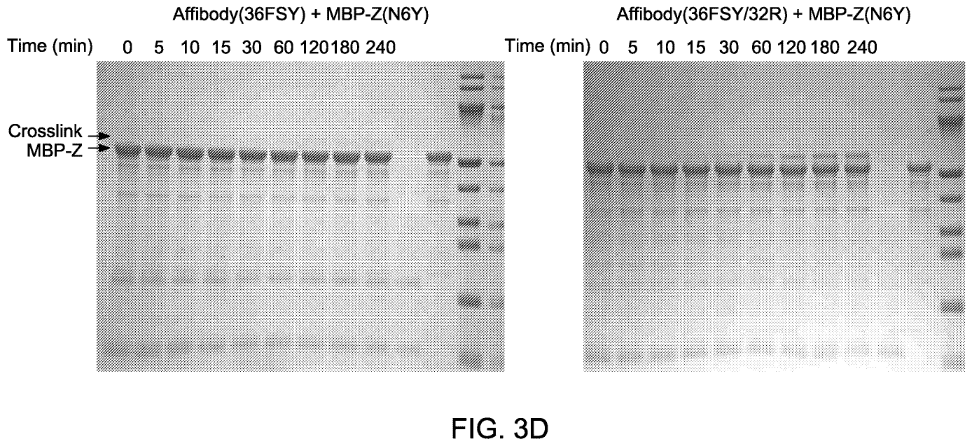

The inventors tested Arg's acceleration effect in affibody. The ZSPA affibody-Z protein pair, which binds with a relatively low affinity (Kd=6 μM), was used. On the basis of the crystal structure of the affibody-Z complex, FSY was incorporated at site D36 and F32 was mutated into Arg in the affibody (FIG. 3A). The Z protein was fused with MBP with residue Asn6 mutated into Lys, His or Tyr for reaction with FSY. These mutant affibody and Z proteins were expressed and purified from E. coli, and then incubated in PBS buffer for crosslinking (concentration 10 μM for each). The reaction mixture was analyzed with SDS-PAGE to determine crosslinking extent. As shown in FIGS. 3B-3D, in comparison with the control affibody, introduction of Arg close to FSY accelerated its crosslinking reaction with His, Lys, and Tyr, respectively. These data indicated that Arg accelerated FSY crosslinking regardless the identity of the target residue (His, Lys, or Tyr).

### Example 4

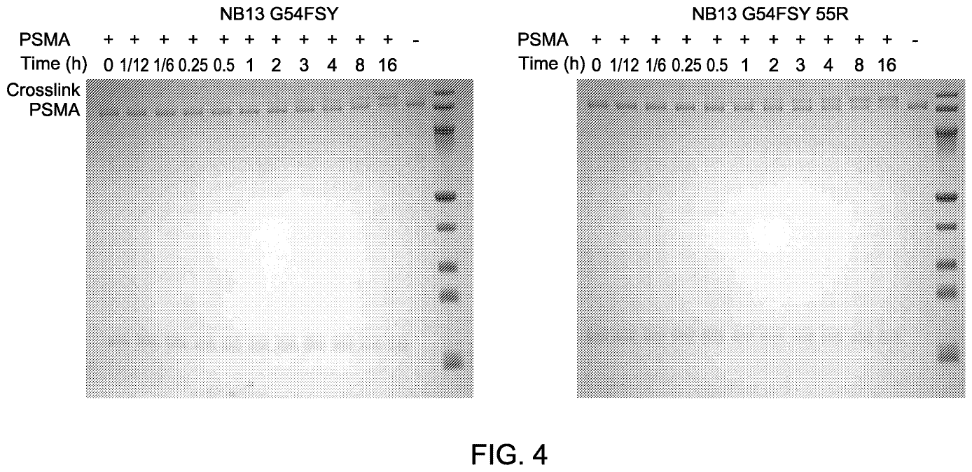

The inventors used Nb13 that is specific for PSMA (prostate-specific membrane antigen) and discovered that FSY incorporation at site G54 of Nb13 crosslinked with PSMA. When residue 55 was mutated to Arg, the resultant mutant Nb13 (G54FSY/55R) exhibited faster crosslinking than Nb12 (G54FSY), as shown in FIG. 4. In these experiments, 3 μM of Nb13 protein was incubated with 0.3 μM of PSMA for different time followed with SDS-PAGE analysis under denatured conditions.

### Example 5

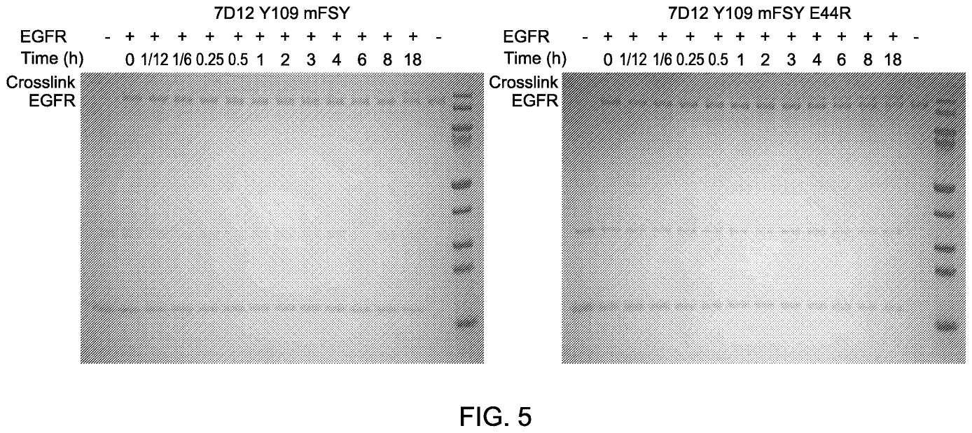

The 7D12 nanobody specific for EGFR was used here. mFSY has the fluorosulfonate warhead at the meta position, in contrast to the para-positioned fluorosulfonate in Uaa FSY. See Klauser et al, Chemical communications 2022, 58 (48), 6861-6864. mFSY was incorporated at site Y109, and mutated E44 to Arg. 3 μM of 7D12 nanobody protein was incubated with 0.3 μM of EGFR ECD protein for different time followed with SDS-PAGE analysis under denatured conditions. For 7D12 (Y109mFSY), crosslinking was detectable after 4 h incubation. For Arg mutant 7D12 (Y109mFSY/E44R), crosslinking could be detected after 1 h incubation. FIG. 5.

### Example 6

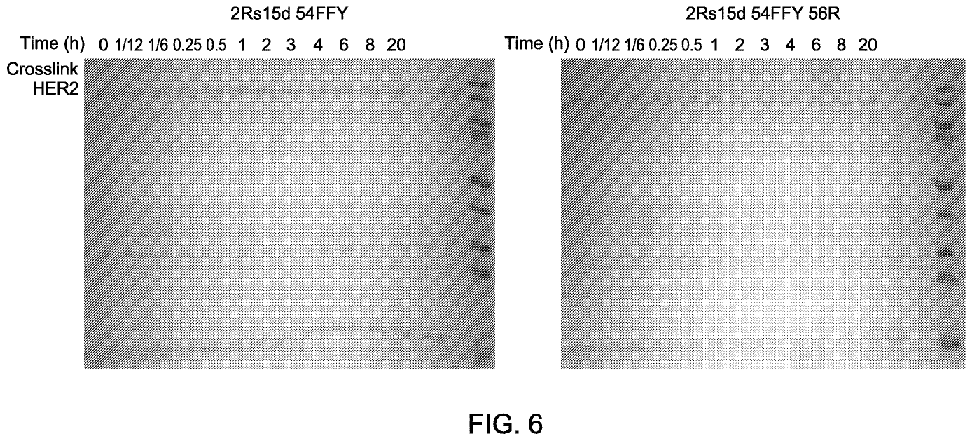

FFY possess faster crosslinking rate than FSY (~2.4 fold increase). See Yu et al, Chem 2022, 8 (10), 2766-2783. Here we found that Arg could further accelerate FFY-mediated protein crosslinking. Nanobody 2Rs15d specific for HER2 was used for testing. FFY was incorporated at site 54 in nanobody 2Rs15d and mutated residue D56 to Arg. 3 mM of 2Rs 15d nanobody protein was incubated with 0.3 mM of HER2 ECD protein for different time followed with SDS-PAGE analysis under denatured conditions. While 2Rs15d (54FFY) could rapidly crosslink in 10 minutes, the Arg mutant 2Rs15d (54FFY/D56R) showed even faster crosslinking within 5 minutes. FIG. 6.

### Example 7

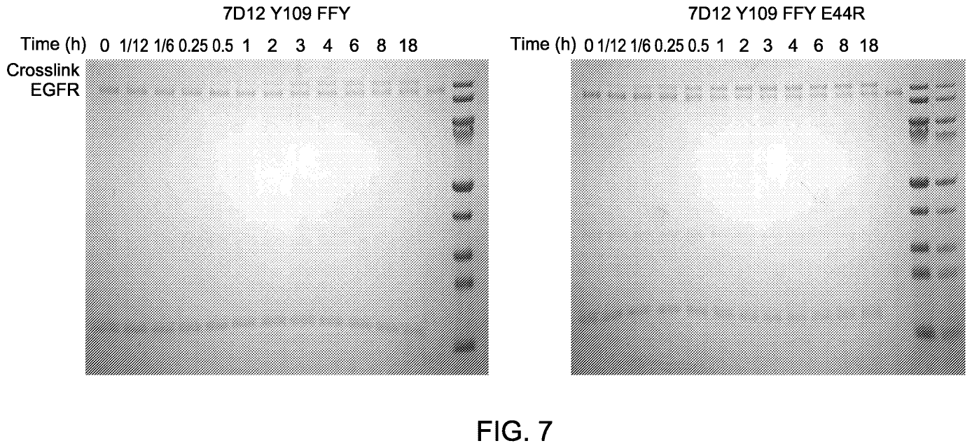

Acceleration of FFY-mediated crosslinking was also verified in nanobody 7D12 specific for the EGFR receptor. FFY was incorporated into 7D12 at site 109 and E44 residue was mutated to Arg. 3 μM of 7D12 nanobody protein was incubated with 0.3 μM of EGFR ECD protein for different time followed with SDS-PAGE analysis under denatured conditions. As can be seen in FIG. 7, while nanobody 7D12 (Y109FFY) crosslinked HER2 ECD in 30 min, the Arg mutant 7D12 (Y109FFY/E44R) was able to crosslink HER2 ECD within 5 min.

### Example 8

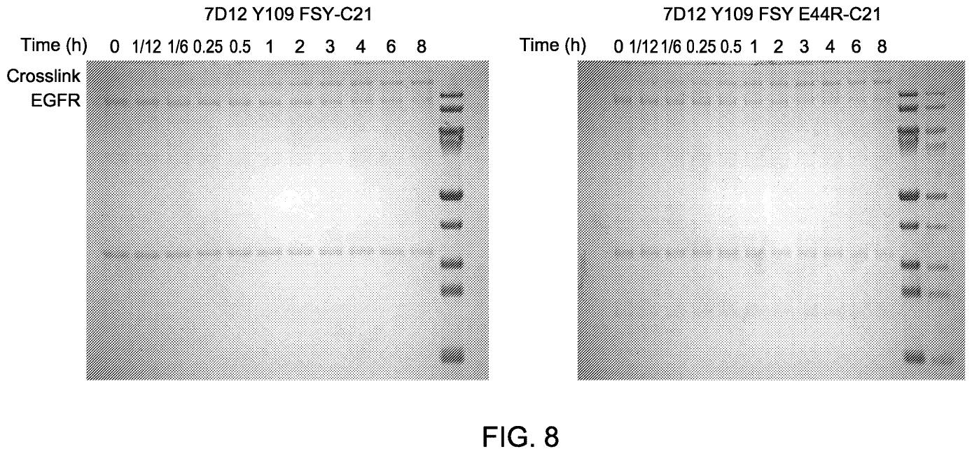

Arg mutation enhances the biological function of a bispecific NK-cell engager (BiKE). To demonstrate the utility of Arg acceleration of protein crosslinking via SuFEx reaction, we evaluated if Arg mutation could enhance the biological function of a bispecific NK-cell engager (BiKE). A BiKE specific for EGFR and NK cells was generated by fusing nanobody 7D12 (specific for EGFR) with nanobody C₂₁ (specific for CD16 on NK). FSY was incorporated at site Y109 of 7D12 to enable BiKE crosslinking with EGFR, and Arg mutation was introduced at site E44 of 7D12 to accelerate the crosslinking. We first compared the crosslinking of EGFR ECD protein with 7D12 (Y109FSY)-C₂₁ (WT BiKE) and 7D12 (Y109FSY/E44R)-C₂₁ (Arg-BiKE). As shown in FIG. 8, the Arg-BiKE crosslinked EGFR ECD in 5 min, while the WT-BiKE crosslinked EGFR ECD in 30 min, confirming Arg acceleration effect in this BiKE.

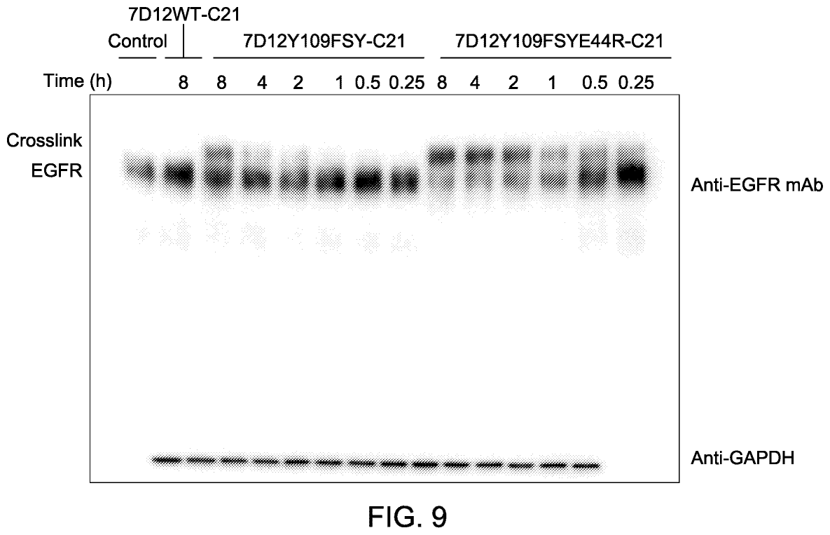

Next, we verified that both WT BiKE and Arg-BiKE were able to crosslink the EGFR on the surface of A431 cancer cell line. 1 nM of the BiKE proteins were incubated with the EGFR+A431 cells for different time, after which the cells were lysed and cell lysate analyzed by Western blot to detect crosslinking. As expected, the Arg-BiKE crosslinked EGFR on cells swiftly within 15 min, while the WT-BiKE needed 2 h to show detectable crosslinking (FIG. 9).

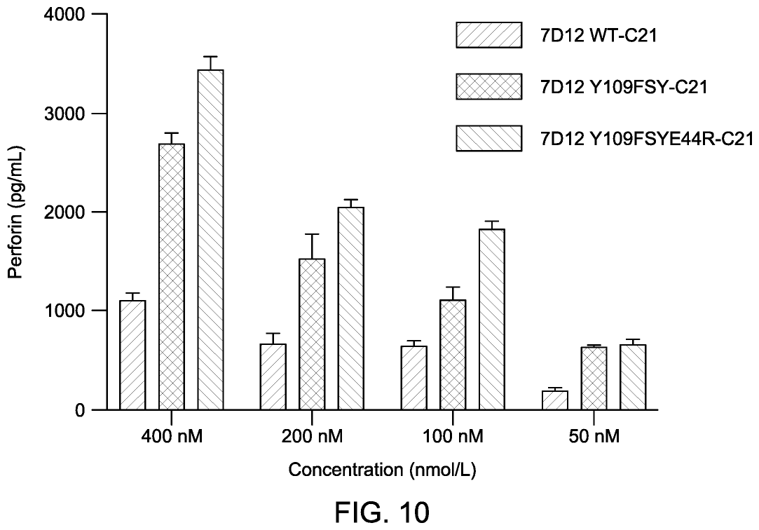

To compare the Arg-BiKE and WT-BiKE in activating NK toward EGFR-expressing cells, the EGFR+A431 cells (10K) were incubated with different concentrations of BiKE (50 nM, 100 nM, 200 nM, 400 nM) for 1 h, followed with washing to remove the BiKE (10 min each for 3 times). NK cells (100K) were then added and incubated with the treated A431 cells for 4 h. The supernatant of the cells was assayed with the human perforin ELISA kit to measure perforin, a marker for NK cell activity. As shown in FIG. 10, the FSY incorporated WT-BiKE and Arg-BiKE both increased perforin release from NK cells when compared to the noncovalent parental BiKE that had no FSY incorporation. In addition, the Arg-BiKE exhibited significant increase in activating NK release of perforin than the WT-BiKE when BiKE concentration was greater than 100 nM. These results indicate that Arg acceleration of SuFEx reaction in proteins is beneficial for the development of covalent protein therapeutics.

### Example 9

Tetramethylguanidine (TMG) has no effect on protein crosslinking via SuFEx reaction. TMG has been frequently used as a catalyst to accelerate the SuFEx reaction between small molecules (Homer et al, Nat Rev Methods Primers 2023, 3 (1), 58. doi.org/10.1038/s43586-023-00241-y). The inventors found that TMG had no effect on SuFEx crosslinking in the protein context. 20 mM of TMG was added into all protein pairs described in the examples above, followed with SDS-PAGE analysis of the crosslinking efficiency. There was no detection of any acceleration of the reaction rate or increase in the crosslinking yield by TMG (data not shown).

### Example 10

Methods of incorporating unnatural amino acid side chains, such as those described herein, into proteins are known in the art and described, for example, in US Publication No. 2020/010411, US Publication No. 2021/002325, US Publication No. 2022/107327, US Publication No. 2022/371986, and WO 2022/232377, the disclosures of each of which are incorporated by reference herein in their entirety.

References: (1) Li et al, Cell 2020, 182 (1), 85-97.e16; (2) Yu et al, Chem 2022, 8 (10), 2766-2783; (3) Wang et al, J. Am. Chem. Soc. 2018, 140 (15), 4995-4999; (4) Klauser et al, Chemical communications 2022, 58 (48), 6861-6864; (5) Liu et al, J. Am. Chem. Soc. 2021, 143 (27), 10341-10351; (6) Sun et al, Nature Chemistry 2022, 15 (1), 21-32; (7) Li et al, Nature Chemistry 2022, 15 (1), 21-32.

### Informal Sequence Listing

Nanobody 7D12 (Nb_{EGFR})
SEQ ID NO: 1
QVKLEESGGG SVQTGGSLRL TCAASGRTSR SYGMGWFRQA PGKEREFVSG
ISWRGDSTGY ADSVKGRFTI SRDNAKNTVD LQMNSLKPED TAIYYCAAAA
GSAWYGTLYE YDYWGQGTQV TVSS
Nanobody 7D12
SEQ ID NO: 2
QVKLEESGGG SVQTGGSLRL TCAASGRTSR SYGMGWFRQA PGKRREFVSG
ISWRGDSTGY ADSVKGRFTI SRDNAKNTVD LQMNSLKPED TAIYYCAAAA
GSAWYGTLX^{FSY}E YDYWGQGTQV TVSS
SEQ ID NO: 3
Nanobody 7D12 CDR1 = RTSRSYGMG
SEQ ID NO: 4
Nanobody 7D12 CDR2 = GISWRGDS
SEQ ID NO: 5
Nanobody 7D12 CDR3 = AAGSAWYGTLYEYDY
SEQ ID NO: 6
Nanobody 7D12 CDR3 = AAGSAWYGTLX^{FSY}EYDY
SEQ ID NO: 7
Nanobody 7D12 Framework Region = WFRQA PGKRREFVS
Nanobody 2rs15d (Nb_{Her2})
SEQ ID NO: 8
QVQLQESGGG SVQAGGSLKL TCAASGYIFN SCGMGWYRQS PGRERELVSR
ISGDGDTWHK ESVKGRFTIS QDNVKKTLYL QMNSLKPEDT AVYFCAVCYN
LETYWGQGTQ VTVSS
Nanobody 2rs15d (Nb_{Her2})
SEQ ID NO: 9
QVQLQESGGG SVQAGGSLKL TCAASGYIFN SCGMGWYRQS PGRERELVSR
ISGX^{FSY}GRTWHK ESVKGRFTIS QDNVKKTLYL QMNSLKPEDT AVYFCAVCYN
LETYWGQGTQ VTVSS
SEQ ID NO: 10
Nanobody 2rs15d CDR1 = GYIFNSCG
SEQ ID NO: 11
Nanobody 2rs15d CDR2 = RISGDGD
SEQ ID NO: 12
Nanobody 2rs15d CDR3 = AVCYNLETY
SEQ ID NO: 13
Nanobody 2rs15d CDR2 = RISGX^{FSY}GR
Z_{SPA}
SEQ ID NO: 14
VDNKFNKELS VAGREIVTLP NLNDPQKKAF IFSLWDDPSQ SANLLAEAKK
LNDAQAPK
Z_{SPA}
SEQ ID NO: 15
VDNKENKELS VAGREIVTLP NLNDPQKKAF IRSLWX^{FSY}DPSQ SANLLAEAKK
LNDAQAPK
Nanobody Nb13
SEQ ID NO: 16
QVQLQESGGG SVQTGGSLRL SCAASGYTAS FSWIGYFRQA PGKEREGVAV
INVGVGSTYY ADSVKGRFTI SRDNTENTIS LEMNSLKPED TGLYYCAGSL
RWSRPPNPIS EDAYNYWGQG TQVTVSS

1. A protein comprising:
(i) an unnatural amino acid, and
(ii) a non-naturally occurring arginine;
wherein:
(a) the non-naturally occurring arginine is proximal to the —S(O₂) F group in the unnatural amino acid side chain; and
(b) the unnatural amino comprises a side chain of Formula (I): 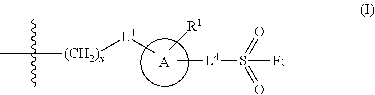
wherein:
ring A is phenyl, a 5-membered cycloalkyl, a 5-membered heterocycloalkyl, or a 5-membered heteroaryl;
L⁴ is a bond or —O—;
x is an integer from 0 to 8;
L¹ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl;
X¹ is independently —F, —Cl, —Br, or —I;
R^{1A} is hydrogen, substituted or unsubstituted alkyl, or substituted or unsubstituted heteroalkyl;
R^{1B} is hydrogen, substituted or unsubstituted alkyl, or substituted or unsubstituted heteroalkyl;
n1 is an integer from 0 to 4;
m1 is 1 or 2; and
v1 is 1 or 2.

2. A protein comprising:
(i) an unnatural amino acid, and
(ii) a naturally occurring arginine;
wherein:
(a) the arginine is proximal to the —S(O₂) F group in the unnatural amino acid side chain; and
(b) the unnatural amino comprises a side chain of Formula (I): 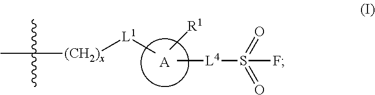
wherein:
ring A is phenyl, a 5-membered cycloalkyl, a 5-membered heterocycloalkyl, or a 5-membered heteroaryl;
L⁴ is a bond or —O—;
x is an integer from 0 to 8;
L¹ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR^{1A}, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl;
X¹ is independently —F, —Cl, —Br, or —I;
R^{1A} is hydrogen, substituted or unsubstituted alkyl, or substituted or unsubstituted heteroalkyl;
R^{1B} is hydrogen, substituted or unsubstituted alkyl, or substituted or unsubstituted heteroalkyl;
n1 is an integer from 0 to 4;
m1 is 1 or 2; and
v1 is 1 or 2;
provided that when A is phenyl, then the protein is not epidermal growth factor receptor, protein tyrosine phosphatase 1B, P-selectin glycoprotein ligand 1, complement component 5a receptor, chemokine receptor D6, CXCR4, thymopentin, oxytocin, arginine vasopressin, indolicidin, or a protein described in any one of WO 2017/161183, WO 2019/173760, WO 2020/072674, WO 2020/206341, and WO 2022/232377.

3-119. (canceled)

120. The protein of claim 1, wherein R¹ is ortho or meta to —S(O₂)F.

121. The protein of claim 1, wherein the unnatural amino comprises:
(a) a side chain of Formula (II): 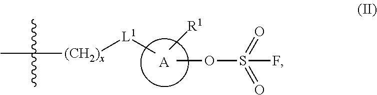
(b) a side chain of Formula (V): . 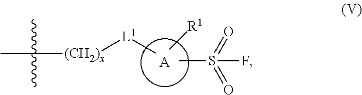
(c) a side chain of Formula (III): 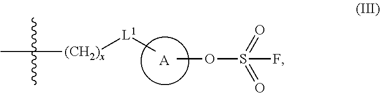
or
(d) a side chain of Formula (VI):

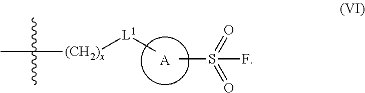

122. The protein of claim 1, wherein ring A is pyrrole, pyrazole, imidazole, triazole, furan, thiophene, phosphole, oxazole, isoxazole, thiazole, or isothiazole; L¹ is a bond —NH—C(O)—(CH₂)_{y}—, —NH—C(O)—O—(CH₂)_{y}—, —NH—C(O)—NH—(CH₂)_{y}—, —NH—C(O)—S—(CH₂)_{y}—, and y is an integer from 0 to 2.

123. The protein of claim 1, wherein the unnatural amino comprises:
(a) a side chain of Formula (FSY): 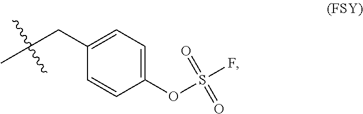 [SMILES: *Cc1ccc(OS(=O)(=O)F)cc1]
(b) a side chain of Formula (mFSY): 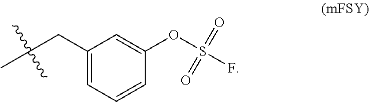 [SMILES: *Cc1cccc(OS(=O)(=O)F)c1]
(c) a side chain of Formula (FFY): 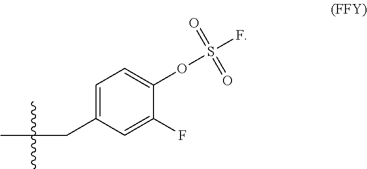 [SMILES: *Cc1ccc(OS(=O)(=O)F)c(F)c1]
(d) a side chain of Formula (FSK): 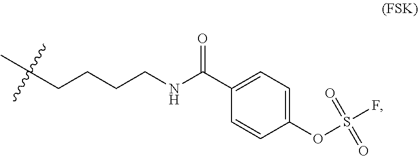 [SMILES: *CCCCNC(=O)c1ccc(OS(=O)(=O)F)cc1]
(e) a side chain of Formula (mFSK): 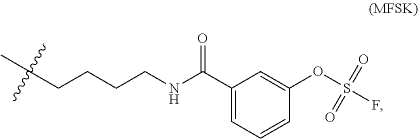 [SMILES: *CCCCNC(=O)c1cccc(OS(=O)(=O)F)c1]
(f) a side chain of Formula (IV): 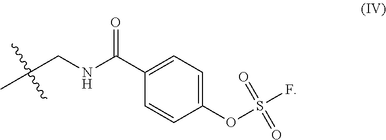 [SMILES: *CNC(=O)c1ccc(OS(=O)(=O)F)cc1]
(g) a side chain of Formula (NHSF): 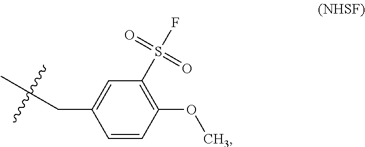 [SMILES: *Cc1ccc(OC)c(S(=O)(=O)F)c1]
(h) a side chain of Formula (VII): 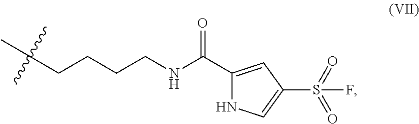 [SMILES: *CCCCNC(=O)c1cc(S(=O)(=O)F)c[nH]1]
(i) a side chain of Formula (VIII): 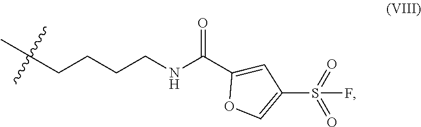 [SMILES: *CCCCNC(=O)c1cc(S(=O)(=O)F)co1]
(j) a side chain of Formula (IX): 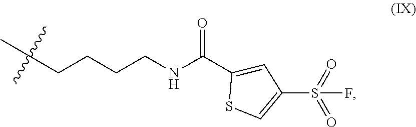 [SMILES: *CCCCNC(=O)c1cc(S(=O)(=O)F)cs1]
or
(k) a side chain of Formula (X):

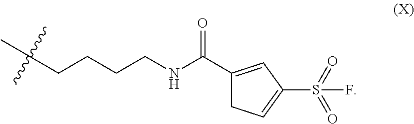 [SMILES: *CCCCNC(=O)C1=CC(S(=O)(=O)F)=CC1]

124. The protein of claim 1, wherein the protein is an antibody or an antibody variant.

125. The protein of claim 124, wherein the antibody variant is a single-chain variable fragment, a single-domain antibody, an affibody, or an antigen-binding fragment.

126. The protein of claim 124, wherein the antibody variant is a single-domain antibody.

127. The protein of claim 1, wherein the protein comprises the amino acid sequence of SEQ ID NO:1, SEQ ID NO:2, SEQ ID NO:8, SEQ ID NO:9, SEQ ID NO: 14, or SEQ ID NO: 15.

128. The protein of claim of claim 1, wherein the unnatural amino acid is within a CDR region of the antibody or antibody variant, and the non-naturally occurring arginine is within a CDR region of the antibody or antibody variant.

129. The protein of claim of claim 1, wherein the unnatural amino acid is within a CDR region of the antibody or antibody variant, and the non-naturally occurring arginine is within a framework region of the antibody or antibody variant.

130. A nucleic acid encoding the protein of claim 1.

131. A vector comprising a nucleic acid encoding the protein of claim 1.

132. A cell comprising: (a) the protein of claim 1, (b) a nucleic acid encoding the protein of claim 1, or (c) a vector comprising a nucleic acid encoding the protein of claim 1.

133. A pharmaceutical composition comprising the protein of claim 1 and a pharmaceutically acceptable excipient.

134. A method of covalently binding a protein to a target protein, the method comprising contacting the protein of claim 1 with a lysine, histidine, or tyrosine of a target protein, thereby covalently binding the protein to the target protein.

135. The method of claim 134, wherein the protein is a cell surface receptor, a cytosolic protein, a transcriptional factor, or an enzyme.

136. The method of claim 134, wherein the target protein is a programmed death-ligand 1 receptor, a programmed cell death protein 1 receptor, a 5-hydroxytryptamine receptor, an acetylcholine receptor, an adenosine receptor, an adenosine A2A receptor, an adenosine A2B receptor, an angiotensin receptor, an apelin receptor, a bile acid receptor, a bombesin receptor, a bradykinin receptor, a cannabinoid receptor, a chemerin receptor, a chemokine receptor, a cholecystokinin receptor, a Class A Orphan receptor, a dopamine receptor, an endothelin receptor, an epidermal growth factor receptor, a formyl peptide receptor, a free fatty acid receptor, a galanin receptor, a ghrelin receptor, a glycoprotein hormone receptor, a gonadotrophin-releasing hormone receptor, a G protein-coupled receptor, a G protein-coupled estrogen receptor, a histamine receptor, a hydroxycarboxylic acid receptor, a kisspeptin receptor, a leukotriene receptor, a lysophospholipid receptor, a lysophospholipid SIP receptor, a melanin-concentrating hormone receptor, a melanocortin receptor, a melatonin receptor, a motilin receptor, a neuromedin U receptor, a neuropeptide FF/neuropeptide AF receptor, a neuropeptide S receptor, a neuropeptide W/neuropeptide B receptor, a neuropeptide Y receptor, a neurotensin receptor, an opioid receptor, an opsin receptor, an orexin receptor, an oxoglutarate receptor, a P2Y receptor, a platelet-activating factor receptor, a prokineticin receptor, a prolactin-releasing peptide receptor, a prostanoid receptor, a proteinase-activated receptor, a QRFP receptor, a relaxin family peptide receptor, a somatostatin receptor, a succinate receptor, a tachykinin receptor, a thyrotropin-releasing hormone receptor, a trace amine receptor, a urotensin receptor, or a vasopressin receptor.

137. A method of enhancing the bioreactivity or binding efficacy of a protein, the method comprising: (i) mutating a first amino acid to an unnatural amino acid, and (ii) mutating a second amino acid proximal to the first amino acid to arginine; wherein the unnatural amino comprises a side chain of Formula (I) 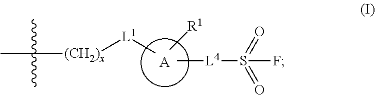
wherein:
ring A is phenyl, a 5-membered cycloalkyl, a 5-membered heterocycloalkyl, or a 5-membered heteroaryl;
L⁴ is a bond or —O—;
x is an integer from 0 to 8;
L¹ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1A}, —SOᵥ₁NR^{1A}R^{1B}, —NHC(O)NR^{1A}R^{1B}, —N(O)ₘ₁, —NR^{1A}R^{1B}, —C(O)R^{1A}, —C(O)—OR^{1A}, —C(O)NR^{1A}R^{1B}, —OR14, —NR^{1A}SO₂R^{1B}, —NR^{1A}C(O)R^{1B}, —NR^{1A}C(O)OR^{1B}, —NR^{1A}OR^{1B}, substituted or unsubstituted alkyl, or substituted or unsubstituted heteroalkyl;
X¹ is independently —F, —Cl, —Br, or —I;
R^{1A} is hydrogen, substituted or unsubstituted alkyl, or substituted or unsubstituted heteroalkyl;
R^{1B} is hydrogen, substituted or unsubstituted alkyl, or substituted or unsubstituted heteroalkyl;
n1 is an integer from 0 to 4;
m1 is 1 or 2; and
v1 is 1 or 2.
